(19) United States
(12) Patent Application Publication
(10) Pub. No.: US 2023/0296606 A1
(43) Pub. Date: Sep. 21, 2023

(54) METHOD FOR THE PROGNOSIS OF DISEASE PROGRESSION IN A PATIENT THAT SUFFERS FROM OR IS AT RISK OF DEVELOPING CANCER

(71) Applicant: BERGONIÉ INSTITUTE, Bordeaux (FR)

(72) Inventors: Antoine ITALIANO, Bordeaux (FR); François LE LOARER, Bordeaux (FR)

(21) Appl. No.: 18/016,488

(22) PCT Filed: Jul. 19, 2021
(86) PCT No.: PCT/EP2021/070141

§ 371 Date: Jan. 17, 2023

(30) Foreign Application Priority Data
Jul. 17, 2020 (EP) 20186400.6

(51) Int. Cl. G01N 33/574; G01N 33/50; G01N 33/563; C12Q 1/6813; C12Q 1/686

(52) U.S. Cl. CPC G01N 33/574; G01N 33/5052; G01N 33/563; C12Q 1/6813; C12Q 1/686; G01N 2800/7028

(57) ABSTRACT

The present invention relates to an in vitro method for the prognosis of disease progression in a patient that suffers from or is at risk of developing cancer, said method comprising the step of determining, in a sample from said patient which is suspected to comprise neoplastic or cancerous tissue, whether or not the sample comprises a tertiary lymphoid structure (TLS), and prognosing disease progression, wherein the prognosis is an assessment regarding the likelihood of improved survival of the patient, and/or better clinical outcome of an immunotherapy.

### FIELD OF THE INVENTION

The present application relates to an in vitro method for the prognosis of disease progression in a patient that suffers from or is at risk of developing cancer

### BACKGROUND

The treatment of neoplastic diseases is still an unsolved problem in modern society. This applies as well for tools and approaches to determine the appropriate therapeutic modalities for a given cancer type, to increase patient survival and quality of life.

These and other problems are solved by the features of the independent claims of the present invention. The dependent claims disclose embodiments of the invention which may be preferred under particular circumstances. Likewise, the specification discloses further embodiments of the invention which may be preferred under particular circumstances.

### BRIEF DESCRIPTION OF THE DRAWINGS

FIG. 1: Principle of immune checkpoint inhibition for cancer therapy.

FIG. 2: Immunohistological detection of TLS by detection of CD3/CD20 in PD-L1 negative NSCLC tissue. FIG. 2A: CD3/CD20 stainings—(CD3, brown; CD20, purple), FIG. 2B: PD-L1/CD8 stainings—(CD8, brown; PDL1, purple).

FIG. 3: Immunohistological detection of TLS CD3/CD20 in PD-L1 positive colorectal cancer tissue. FIG. 3A: CD3/CD20 expression, FIG. 3B: PD-L1/CD8 stainings—(CD8, brown; PDL1, purple). PDL1 positivity according to CPS score.

FIG. 4: Immunohistochemical analysis of a patient suffering from NSCLC, and two CT scans of the same patient. A long-lasting partial response (>29 months) could be shown in a 62-years old male presenting a TLS+/PD-L1− NSCLC. FIG. 4A shows presence of TLS in the NSCLC tissue as indicated by CD3/CD20 immunohistochemistry. FIG. 4B shows absence of PD-L1 expression TPS and CPS negative) in the NSCLC tissue. FIG. 4C shows a CT scan of the patient's upper body at the onset of treatment with the PD-L1 inhibitor Pembrolizumab (2 mg/kg, Once every three weeks). Large tumor lesions can be seen in the CT scan (arrows). FIG. 4D shows a CT scan of the same patient's upper body after 42 days. The tumor lesions are significantly reduced (14 mm to 8 mm diameter).

FIG. 5: A: Illustration of an immunohistological-based detection of TLS at low magnification (upper figure) and high magnification (lower figure). B: illustration of a tissue segmentation obtained after machine learning and image analysis step highlighting TLS and tumor regions. Tumor region surface is 21.2 mm² while TLS count is 4 thus giving a TLS density of 0.19 TLS/mm².

FIG. 6: Determination of the thresholding value for the density of TLS in a cohort of 332 cancer patients treated with immune checkpoint inhibitors. The clinical endpoint was progression-free survival. The figure highlights an optimal cut-off value of 0.023 TLS/mm².

FIGS. 7-9: Median progression free survival (PFS) and overall survival (OS) with different tumor types (“pan tumors”) having different TPC/CSP scores, depending on TLS density (high: upper curve, low: lower curve). TLS density cutoff: 0.023 TLS/mm².

FIGS. 7-12: Median progression free survival (PFS) and overall survival (OS) with Non Small Cell Lung cancer (NSCLC) having different TPC/CSP scores, depending on TLS density (high: upper curve, low: lower curve). TLS density cutoff: 0.023 TLS/mm².

FIGS. 14-16: Median progression free survival (PFS) and overall survival (OS) with different tumor types (“pan tumors”) having different TPC/CSP scores, depending on TLS density (high: upper curve, low: lower curve). Mature TLS density cutoff 0.03 TLS/mm².

FIGS. 17-19: Median progression free survival (PFS) and overall survival (OS) with Non Small Cell Lung cancer (NSCLC) having different TPC/CSP scores, depending on TLS density (high: upper curve, low: lower curve). Mature TLS density cutoff 0.03 TLS/mm².

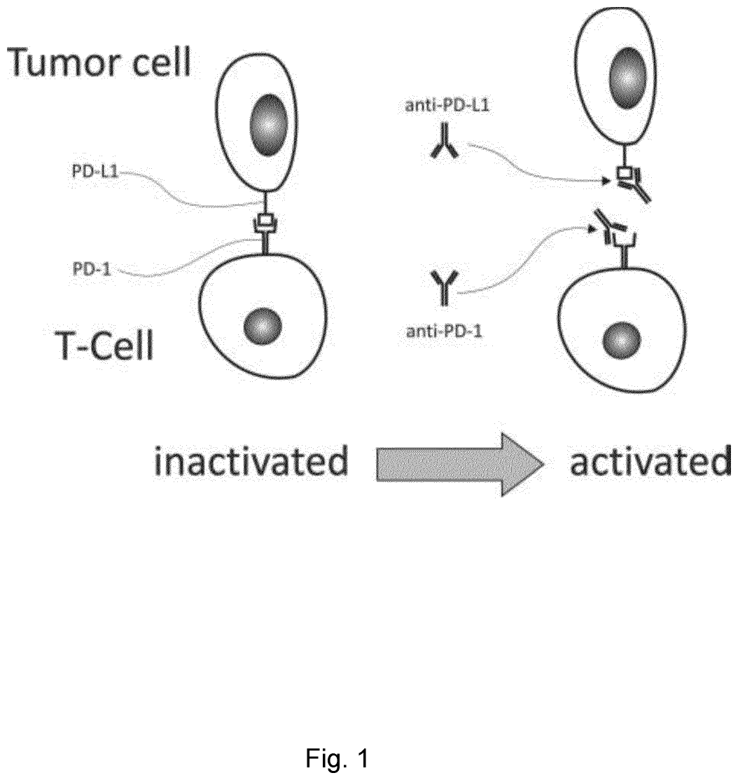

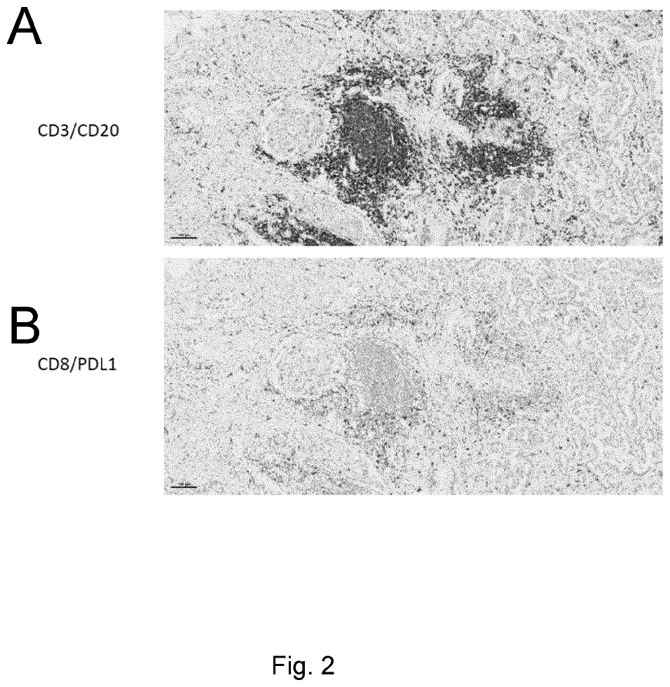

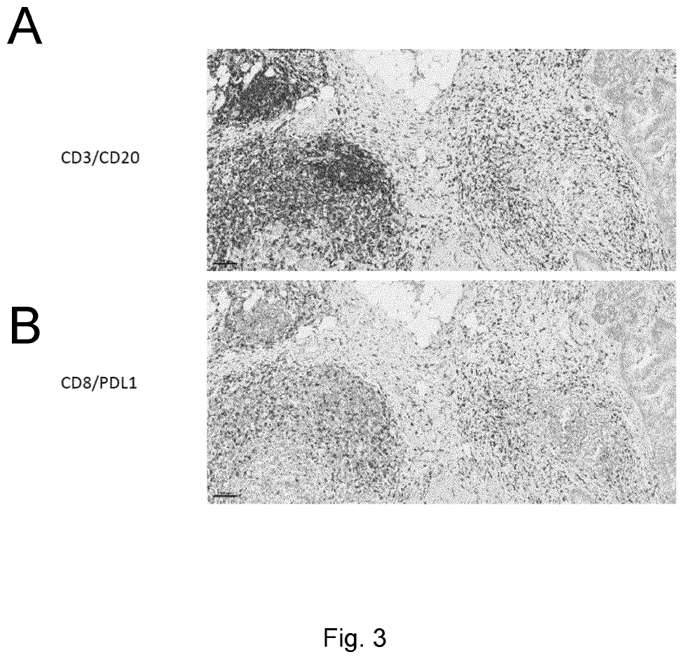

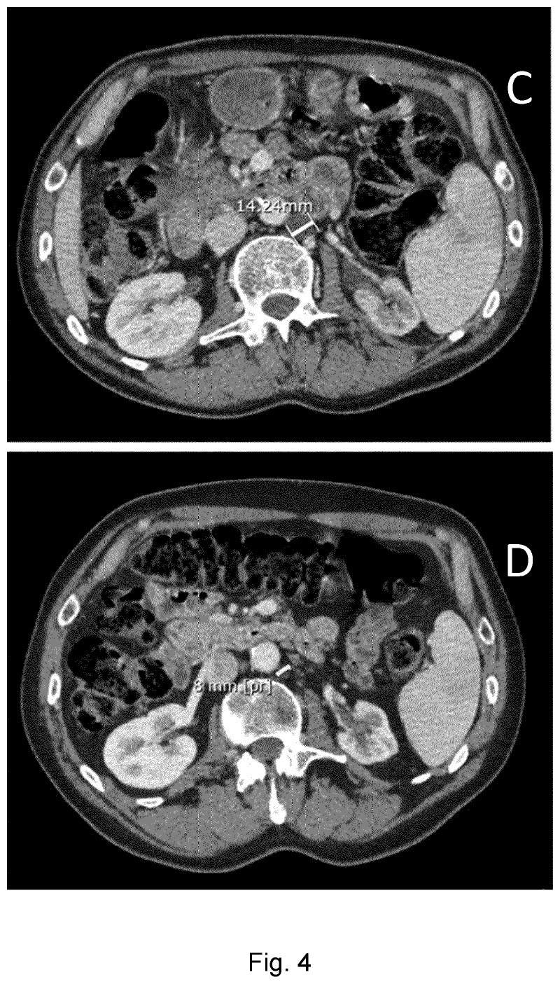

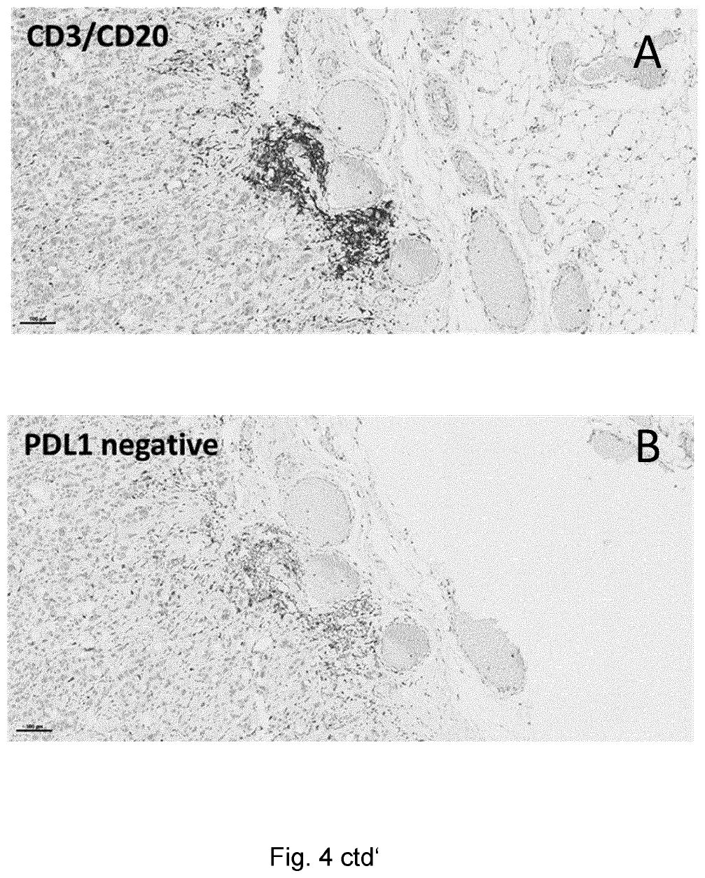

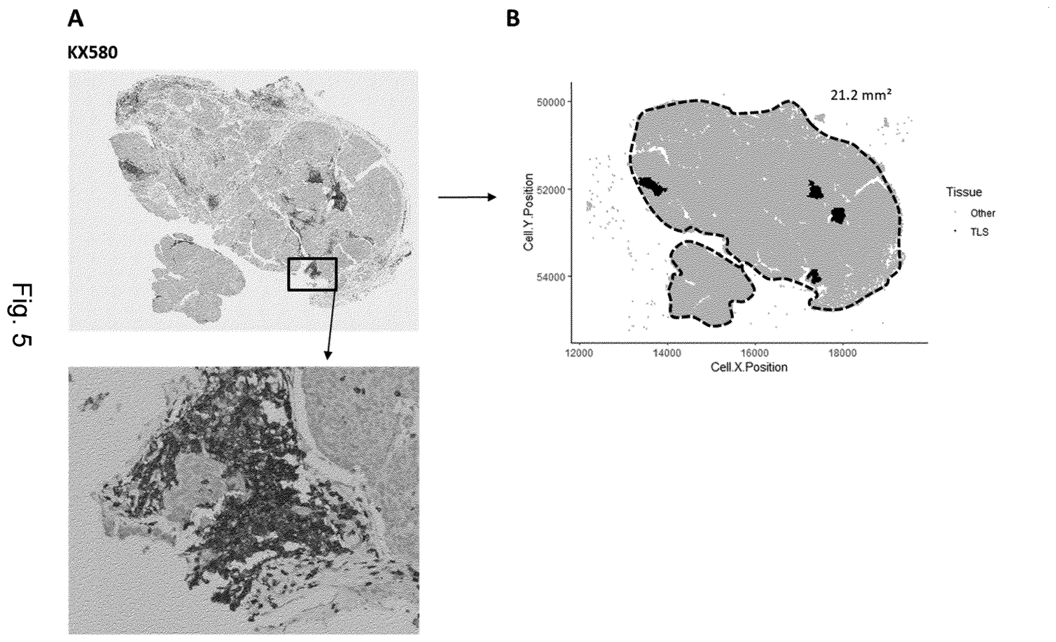

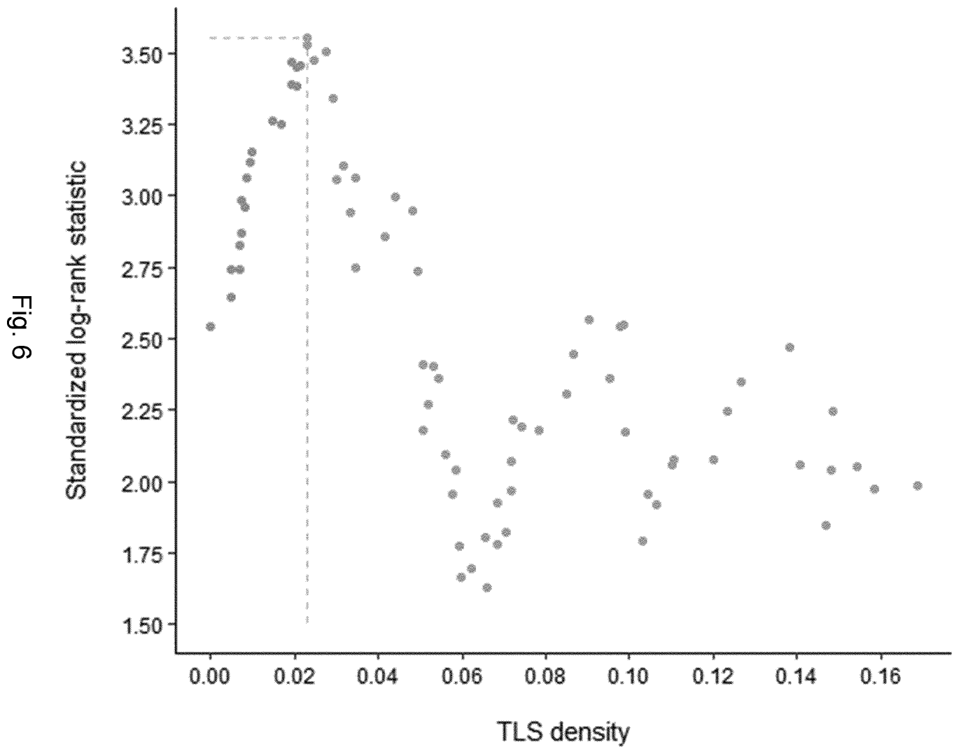

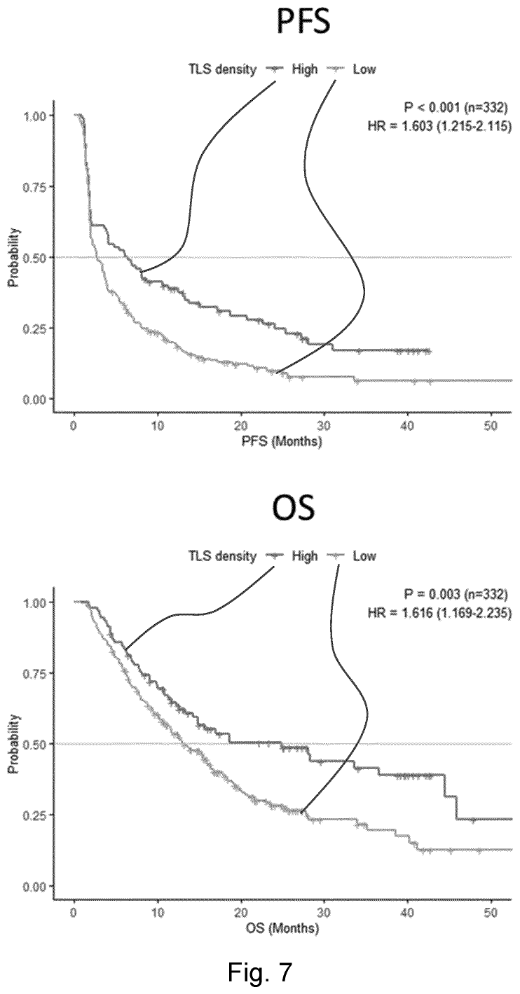

FIG. 7 shows Kaplan Meyer curves, demonstrating that high density of Tertiary Lymphoid Structures (TLS) in tumors (ie. >0.023 TLS/mm²) is associated with improved progression free and overall survival after immune checkpoint therapy, whatever PDL1 expression status (p<0.001 and p=0.003 respectively). The density of mature TLS was calculated for 332 patients according to the ratio of TLS count to the surface of the tumor lesion; i.e. number of mature TLS per mm².

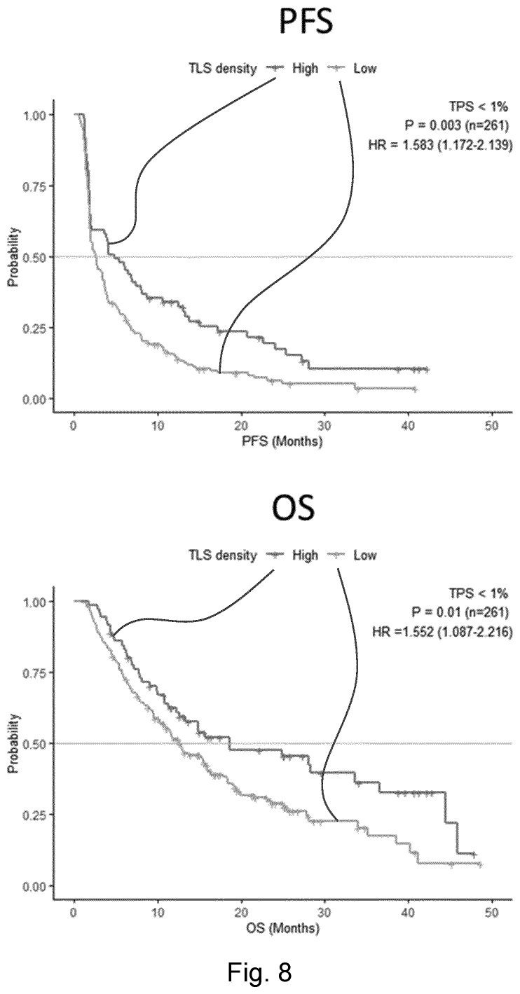

FIG. 8 shows Kaplan Meyer curves, demonstrating that high density of Tertiary Lymphoid Structures (TLS) in tumors (ie. >0.023 TLS/mm²) is associated with improved progression free and overall survival after immune checkpoint therapy, in patients having tumor with TPS score <1% (p=0.003 and 0.01 respectively). The density of mature TLS was calculated for 261 patients according to the ratio of TLS count to the surface of the tumor lesion; i.e. number of mature TLS per mm².

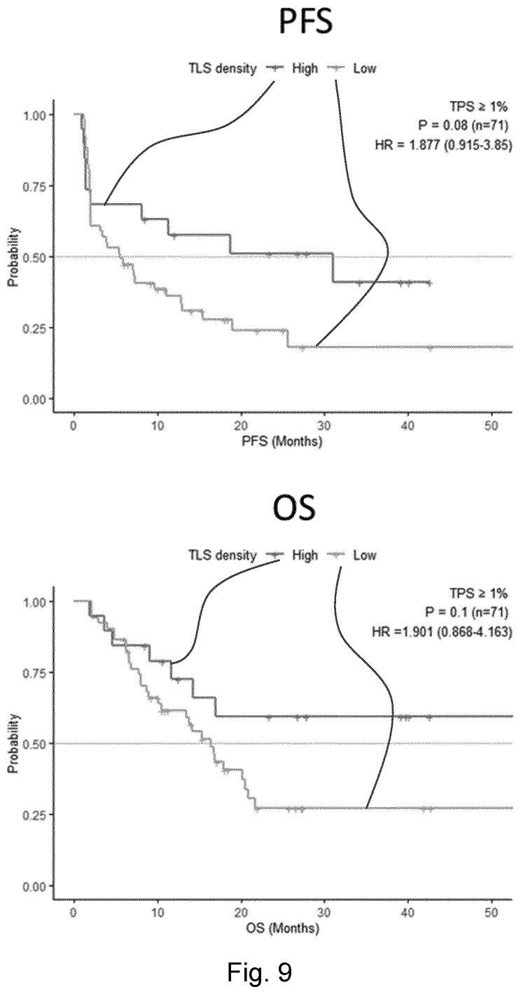

FIG. 9 shows Kaplan Meyer curves, demonstrating that high density of Tertiary Lymphoid Structures (TLS) in tumors (ie. >0.023 TLS/mm²) is associated with improved progression free and overall survival after immune checkpoint therapy, in patients having tumor with TPS score ≥1% (p=0.08 and 0.1 respectively). The density of mature TLS was calculated for 71 patients according to the ratio of TLS count to the surface of the tumor lesion; i.e. number of mature TLS per mm².

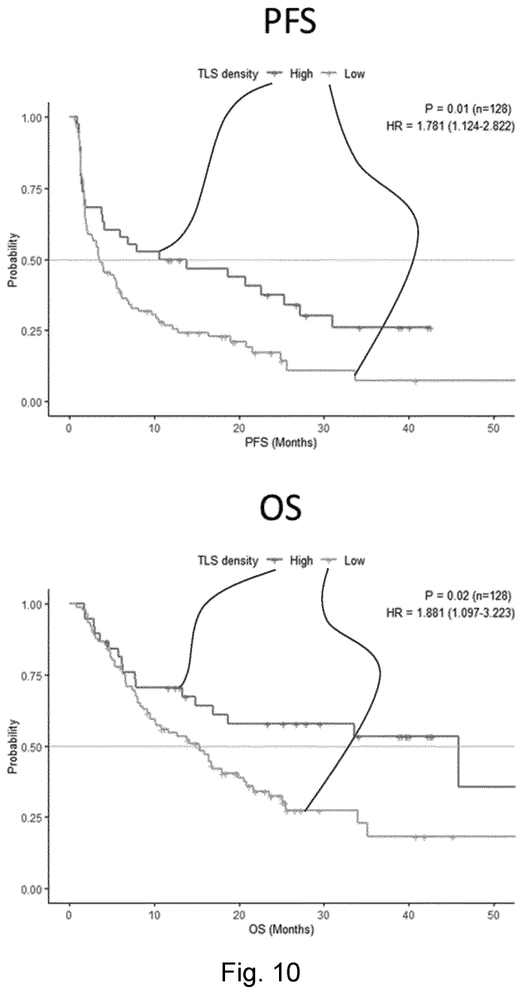

FIG. 10 shows Kaplan Meyer curves, demonstrating that high density of Tertiary Lymphoid Structures (TLS) in tumors (ie. >0.023 TLS/mm²) is associated with improved progression free and overall survival after immune checkpoint therapy in NSCLC patients (p=0.01 and 0.02 respectively), whatever PDL1 expression status. The density of mature TLS was calculated for 128 patients according to the ratio of TLS count to the surface of the tumor lesion; i.e. number of mature TLS per mm².

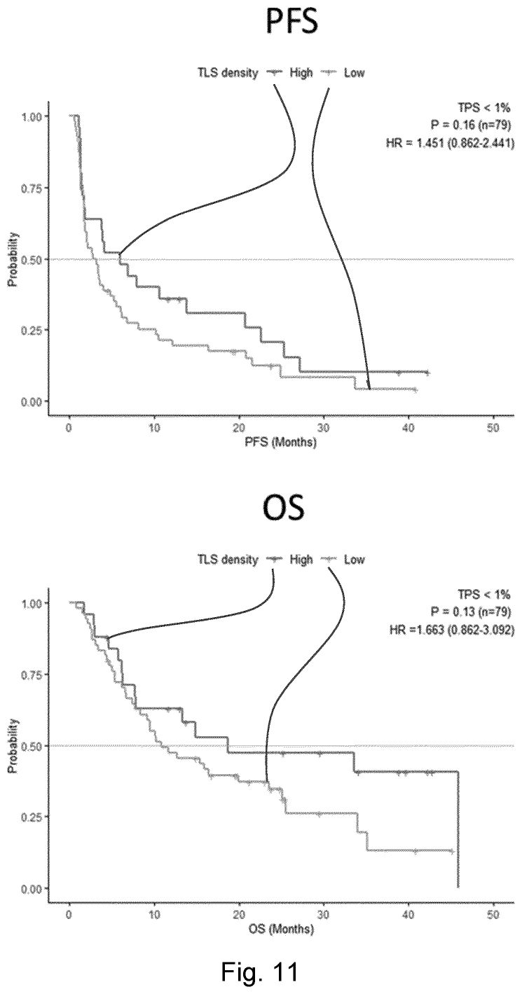

FIG. 11 shows Kaplan Meyer curves, demonstrating that high density of Tertiary Lymphoid Structures (TLS) in tumors (ie. >0.023 TLS/mm²) is associated with improved progression free and overall survival after immune checkpoint therapy in NSCLC patients having tumor with TPS score <1% (p=0.16 and 0.13 respectively). The density of mature TLS was calculated for 79 patients according to the ratio of TLS count to the surface of the tumor lesion; i.e. number of mature TLS per mm².

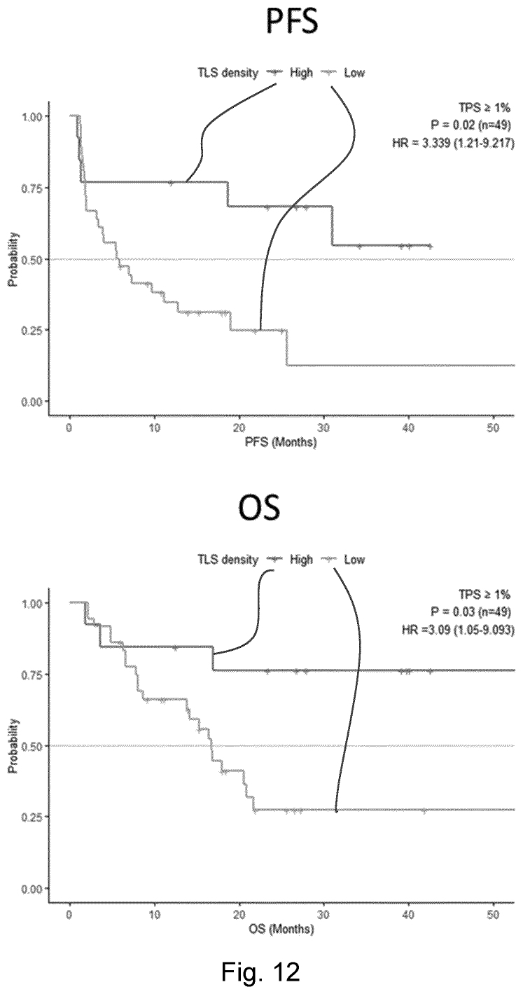

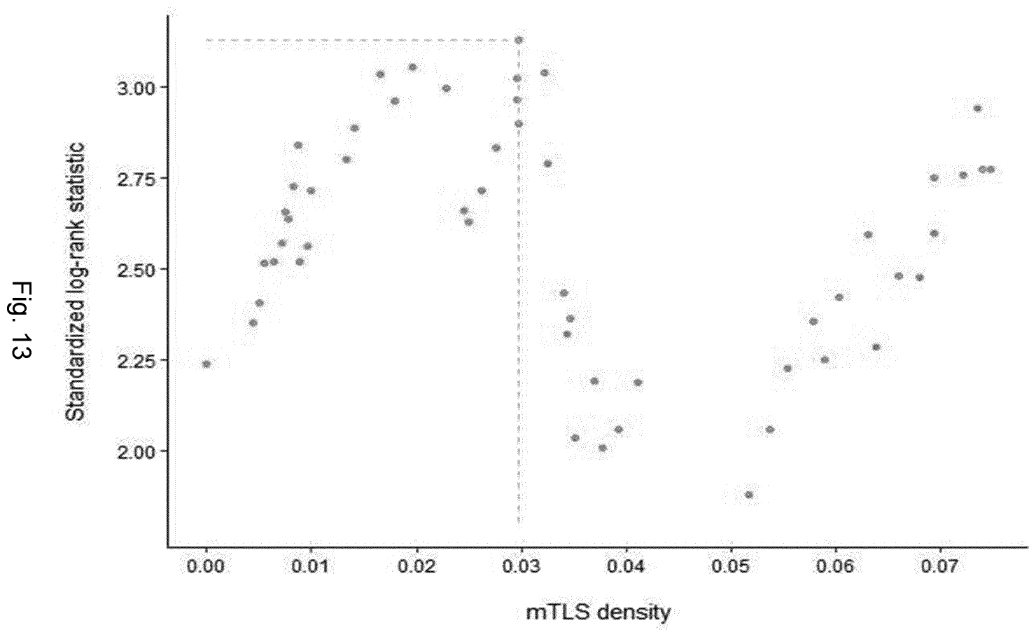

FIG. 12 shows Kaplan Meyer curves, demonstrating that high density of Tertiary Lymphoid Structures (TLS) in tumors (ie. >0.023 TLS/mm²) is associated with improved progression free and overall survival after immune checkpoint therapy in NSCLC patients having tumor with TPS score ≥1% (p=0.02 and 0.03 respectively). The density of mature TLS was calculated for 49 patients according to the ratio of TLS count to the surface of the tumor lesion; i.e. number of mature TLS per mm².

FIG. 13: Determination of the thresholding value for the density of mature TLS in a cohort of 332 cancer patients treated with immune checkpoint inhibitors. The clinical endpoint was progression-free survival. The figure highlights an optimal cut-off value of 0.03 mature TLS/mm².

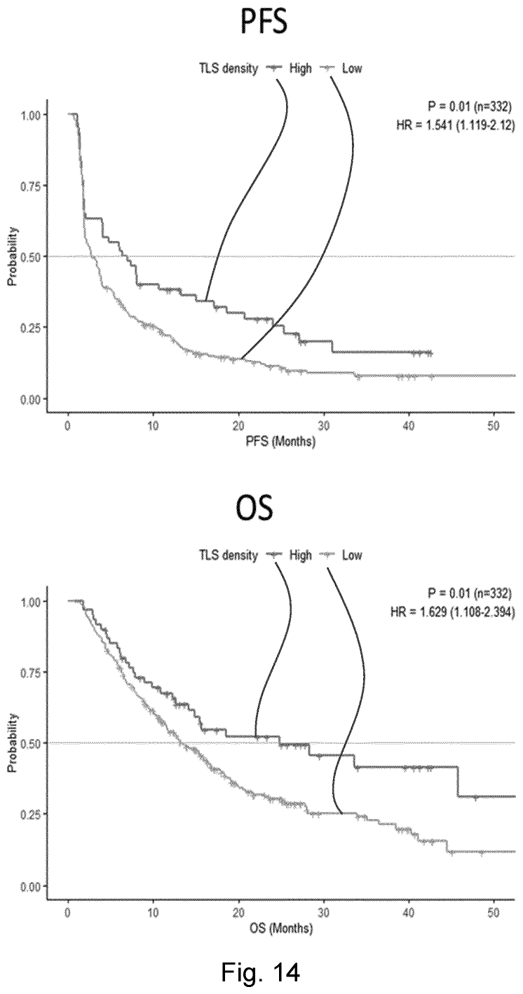

FIG. 14 shows Kaplan Meyer curves, demonstrating that high density of Tertiary Lymphoid Structures (TLS) in tumors (i.e. >0.03 TLS/mm²) is associated with improved progression free and overall survival after immune checkpoint therapy, whatever PDL1 expression status (p=0.01 and p=0.01 respectively). The density of mature TLS was calculated for 332 patients according to the ratio of TLS count to the surface of the tumor lesion; i.e. number of mature TLS per mm².

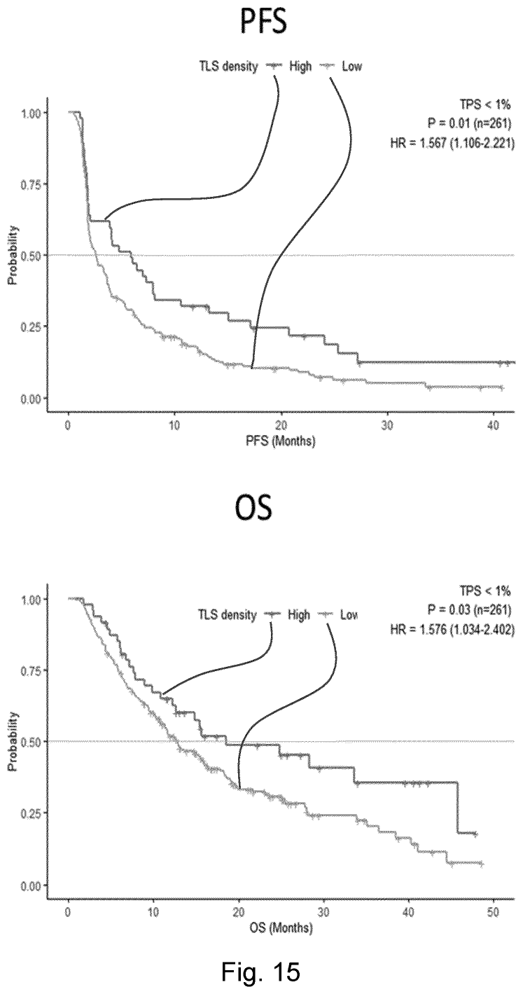

FIG. 15 shows Kaplan Meyer curves, demonstrating that high density of Tertiary Lymphoid Structures (TLS) in tumors (i.e. >0.03 TLS/mm²) is associated with improved progression free and overall survival after immune checkpoint therapy, in patients having tumor with TPS score <1% (p=0.01 and 0.03 respectively). The density of mature TLS was calculated for 261 patients according to the ratio of TLS count to the surface of the tumor lesion; i.e. number of mature TLS per mm².

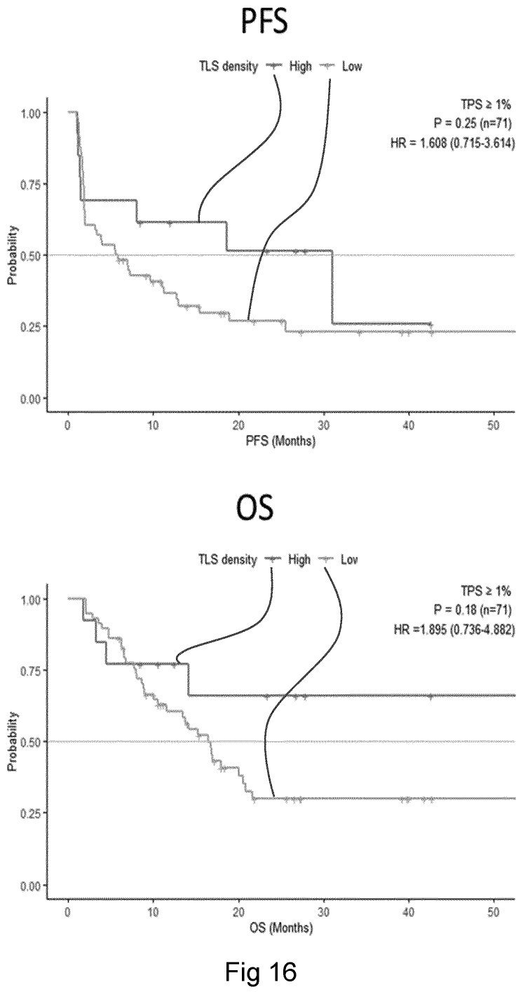

FIG. 16 shows Kaplan Meyer curves, demonstrating that high density of Tertiary Lymphoid Structures (TLS) in tumors (i.e. >0.03 TLS/mm²) is associated with improved progression free and overall survival after immune checkpoint therapy, in patients having tumor with TPS score ≥1% (p=0.25 and 0.18 respectively). The density of mature TLS was calculated for 71 patients according to the ratio of TLS count to the surface of the tumor lesion; i.e. number of mature TLS per mm².

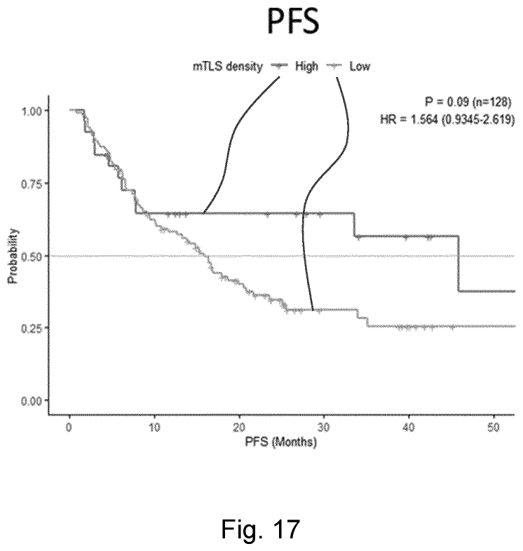

FIG. 17 shows a Kaplan Meyer curve, demonstrating that high density of Tertiary Lymphoid Structures (TLS) in tumors (i.e. >0.03 TLS/mm²) is associated with improved progression free and overall survival after immune checkpoint therapy in NSCLC patients (p=0.09 and 0.1 respectively), whatever PDL1 expression status. The density of mature TLS was calculated for 128 patients according to the ratio of TLS count to the surface of the tumor lesion; i.e. number of mature TLS per mm².

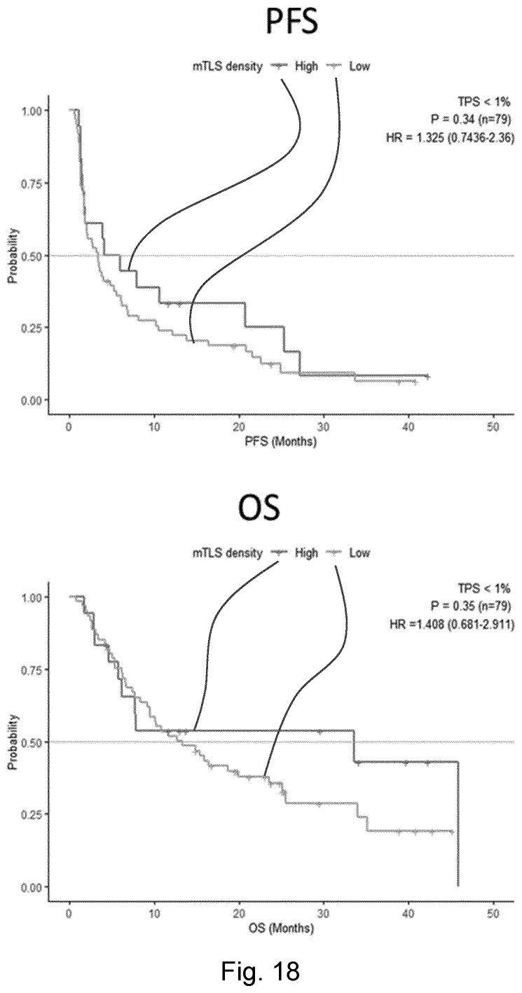

FIG. 18 shows Kaplan Meyer curves, demonstrating that high density of Tertiary Lymphoid Structures (TLS) in tumors (i.e. >0.03 TLS/mm²) is associated with improved progression free and overall survival after immune checkpoint therapy in NSCLC patients having tumor with TPS score <1% (p=0.34 and 0.35 respectively). The density of mature TLS was calculated for 79 patients according to the ratio of TLS count to the surface of the tumor lesion; i.e. number of mature TLS per mm².

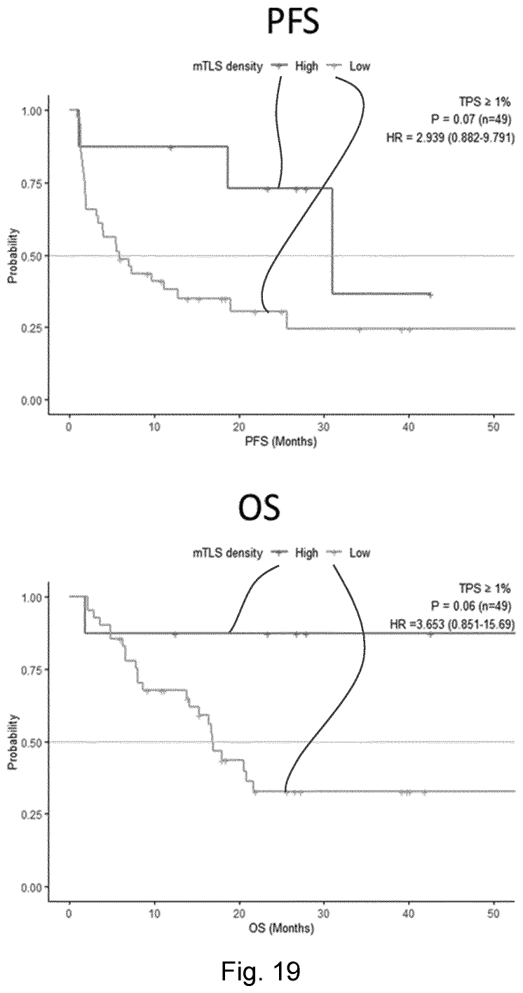

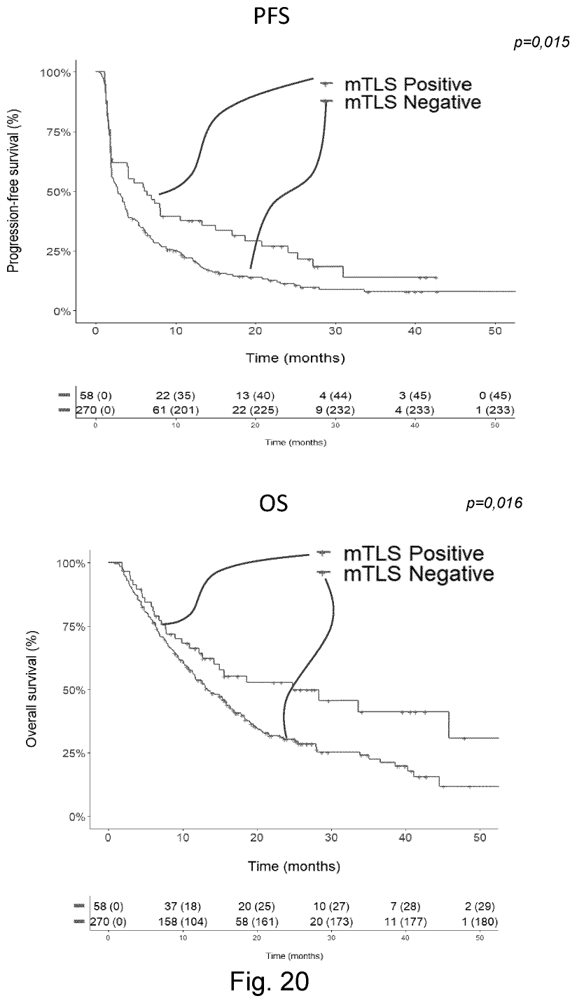

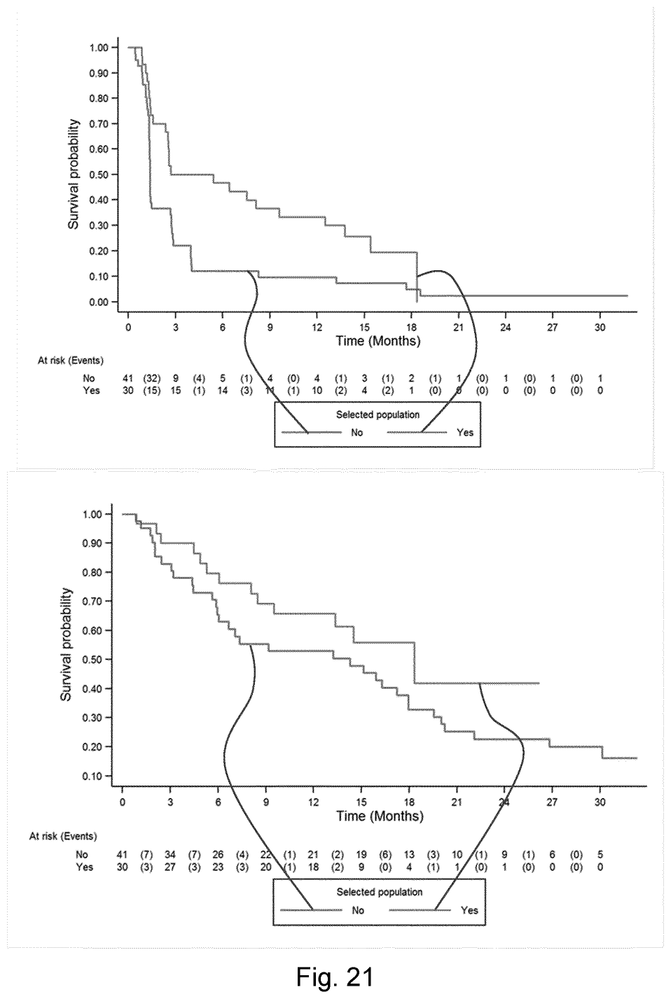

FIG. 19 shows Kaplan Meyer curves, demonstrating that high density of Tertiary Lymphoid Structures (TLS) in tumors (ie. >0.03 TLS/mm²) is associated with improved progression free and overall survival after immune checkpoint therapy in NSCLC patients having tumor with TPS score ≥1% (p=0.07 and 0.06 respectively). The density of mature TLS was calculated for 49 patients according to the ratio of TLS count to the surface of the tumor lesion; i.e. number of mature TLS per mm².

FIG. 20 shows Kaplan Meier curves (PFS & OS) according to the presence or absence of mature TLS in a pan Cancer cohort (338 patients). No cut-off values jave been applied.

FIG. 21 shows Kaplan Meier (PFS & OS) of two independent clinical trials investigating the benefit of immunotherapy (Pembrolizumab, anti-PD1) in patients either selected according to the presence of TLS within the tumor specimen—as assayed by multiplexed-immunohistochemistry and the use of CD3/CD20 markers—(35 patients) or without selection (45 patients). These results highlight the identification of a STS patient sub-population that is more likely to benefit from cancer immunotherapy.

| | TLS cohort | Previous cohorts |
|---|---|---|
| | (n = 35) | (all comers) n = 41 |
| Median Progression-free | 4.1 months | 1.4 months* |
| survival | (CI95%: 2.4-12.5) | (CI95%: 1.3-2.7) |
| 6-months non-progression | 40.0% (22.7-59.4) | 4.9% (0.6-16.5) |
| rate | First endpoint reached | |

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to one aspect of the invention, an in vitro method is provided for the prognosis of disease progression in a patient that suffers from or is at risk of developing cancer, said method comprising the step of:
determining, in a sample from said patient which is suspected to comprise neoplastic or cancerous tissue, whether or not the sample comprises a tertiary lymphoid structure (TLS), and
prognosing disease progression
   wherein the prognosis is an assessment regarding the
likelihood of improved survival of the patient, and/or
better clinical outcome
   of an immunotherapy or treatment with an immunotherapeutic agent.

Tertiary lymphoid structures (TLS) are aggregates of immune cells (mostly B and T cells) that arise in response to immunological stimuli and which have been described to be associated with improved survival in several tumor types. It was recently shown that presence of TLS can be predictive of response to PD1/PDL1 axis blockade in patients with advanced sarcomas (Petitprez et al. Nature 2020)—a finding that has been conjointly corroborated in melanomas and renal cell carcinomas (Cabrita et al. Nature 2020; Helmink et al. Nature 2020).

The term “improved survival”, as used herein, refers to a prolonged period of time during which the subject or patient is alive after treatment with a method described herein. Improved survival denotes the greatest probability of staying free of disease progression for an individual suffering from cancer after a particular treatment. It is also used to describe the high percentage of individuals in a group whose disease is likely to remain stable (without showing signs of progression) after a specific period of time, compared to a control group. It is also used to describe the high percentage of individuals in a group whose disease is likely to cure (without showing signs of disease) after a specific period of time, compared to a control group. This parameter can be measured by any of the usual clinical endpoints indicated as “progression-free survival”, “overall survival” and “disease-free survival” used as an indication of the effectiveness of a particular treatment.

As used herein, the term, “immunotherapeutic agent” relates to a substance or treatment having active or passive immunostimulant activity. Such activity may be a result of specific immunostimulants or non-specific immunostimulants. As used herein, the term “immunotherapy” relates to treatments that may directly or indirectly cause an increase in an immune response or causes an increase in an immune response relative to or in comparison to other therapies.

In one embodiment, such immunotherapy is a cancer immunotherapy. In one embodiment, such immunotherapeutic agent is an agent for cancer immunotherapy.

As used herein, the term “cancer immunotherapy” encompasses the following treatment modalities:

| Modality | Example |
|---|---|
| Cellular immunotherapy | Dendritic cell therapy |
| | CAR-T cell therapy |
| | Adoptive T-cell therapy |
| Antibody therapy | immune checkpoint inhibitor antibodies |
| | Immunoconjugates, i.e., antibodies conjugate |
| | to an immune effector, like e.g. a cytokine |
| | bispecific to also engage T cells (via e.g. a |
| | CD3 binder) or to engage concomitantly two |
| | immune checkpoints (eg. PD1 × 41BB |
| | (CD137); PD1 × CTLA4) |
| | FC engineered to feature increased ADCC |
| T Cell receptor therapy | Soluble T-cell receptors, optionally bispecifc to |
| | also engage T cells (via e.g. a CD3 binder) |
| Cytokine therapy | Interferon |
| | Interleukins |
| Cancer vaccine therapy | Tumor assocated peptides (MHC restricted) |

As used herein, the term “TLS” relates to one or more ectopic lymphoid organs that develop in non-lymphoid tissues at sites of chronic inflammation, including tumors.

Such TLS can be located nearby a tumor, i.e., intra-tumoral, in peritumoral regions, and/or at the invasive front of the tumor. As used herein, the term “stroma” or “tumor stroma” refers to the connective tissue framework and non-tumor cells of a tumor. Examples of some non-tumor cells found in a tumor stroma are fibroblasts and endothelial cells. These cells produce the sum extracellular matrix (ECM) of the tumor. Sometimes, the ECM and the non-malignant cells of the tumor are defined as the “tumor stroma”

As used herein, the term “tumor” means a mass of transformed cells that are characterized, at least in part, by containing angiogenic vasculature. The transformed cells are characterized by neoplastic uncontrolled cell multiplication which is rapid and continues even after the stimuli that initiated the new growth has ceased.

According to one embodiment of the invention, the sample from the patient is a tissue slice. In one embodiment, the sample from the patient is a biopsy. In one embodiment, the sample from the patient is a smear sample. In one embodiment, the sample from the patient is a Fine-needle aspiration (FNA), or sampling (FNS)

In one embodiment, the sample from the patient is a fresh sample. In one embodiment, the sample from the patient is a frozen sample. In still one embodiment, the sample from the patient is an FFPE preserved sample.

According to one embodiment of the invention, the determination of whether or not the sample comprises a tertiary lymphoid structure comprises at least one of
staining the sample with a histochemical staining, and/or
determining the presence or absence of T Lymphocytes and/or B Lymphocytes in the sample.

As used herein, the term “lymphocytes” relates to a type of white blood cells of the immune system. Lymphocytes include natural killer cells, which function in cell-mediated, cytotoxic innate immunity), T cells (for cell-mediated, cytotoxic adaptive immunity), and B cells (for humoral, antibody-driven adaptive immunity). T Lymphocytes and/or B Lymphocytes can be indicative for the presence or absence of TLS.

In one embodiment, the histochemical staining is Hematoxylin and Eosin (H&E) staining.

The detection of the markers disclosed herein can be done on a protein or or mRNA basis.

Detecting a given marker on a protein basis usually involves a method comprising an immunoassay.

Detecting a given marker on a mRNA basis usually involves a method comprising PCR and/or nucleic acid hybridization.

As used herein, the term “a method comprising an immunoassay” relates to a method where an immunological molecule, e.g., an antibody, or a protein having comparable target specific binding characteristics, is used to detect the presence of a target molecule.

In one embodiment, said method comprises IHC (Immunohistochemistry assay), ICC (Immunocytochemistry assay), IF (Immunofluorescence assay) or IHF (Immunohistofluorescence assay).

All these approaches can comprise immunostaining and subsequent image analysis. In this combination of methods the target is stained by means of the immunological molecule or the protein having comparable target specific binding characteristics, namely by direct or indirect labelling of the molecule or protein, and subsequent analysis of the sample by means of video microscopy or other imaging means, and subsequent digital image processing.

In one embodiment, tumor biopsies or FFPE samples are sliced, accordingly treated (e.g., deparaffinized, rehydrated, or subjected to antigen retrieval) and incubated with respective antibodies, that are either labelled or stained with respective labelled antibodies. Detection takes place with suitable imaging methods. Optionally, the slices are counterstained. Image detection and analysis can comprise multispectral image analysis definition of regions of interest, segmentation of tissue into tumor and stroma regions, phenotyping, identification of respective cell types characterized by the respective markers. The density of a given cell type in the sample can be semi-automatically quantified with respective image analysis software (e.g., Inform software, Akoya Bioscience, version 2.4.1). For more details see the experimental description elsewhere herein.

As used herein, the term “a method comprising PCR” relates to a method where polymerase chain reaction is used to detect the presence or absence of a nucleic acid (DNA or mRNA) encoding for a target molecule.

In one embodiment, the in vitro method comprising PCR and/or nucleic acid hybridization comprises at least one of
In situ hybridization (ISH),
In situ PCR,
Real time PCR, and/or
Reverse transcription PCR/qPCR

All these methods are well known to the skilled person. While the former two deliver spatial resolution and can hence be used for density or proximity measurements, the latter two don't and can hence only be used for infiltration measurements, as e.g. of macrophages in to the tumor tissue.

Tumor samples, either fresh, cryopreserved or FFPE, can be used for RNA extraction and gene expression profiling through PCR-based approaches. Among these approaches, RT-qPCR or RNAsequencing or Nanostring can be applied for the assessment of single and/or group of genes expression that can serve as a signature for patient inclusion. High expression of a certain gene or enrichment of a class of gene can constitute a predictive biomarkers. In particular, macrophages related markers can be evaluated.

As used herein, the term “a method comprising nucleic acid hybridization” relates to a method in which nucleic acid probes are used which hybridize with nucleic acids in the sample that are to be determined.

According to one embodiment of the invention, the expression, presence or absence of at least one marker selected from the group consisting of CD3, CD4 and CD8, CD20, CD19 and Pax5 is detected on a protein- or mRNA basis.

Regarding CD3, this molecule consists of three subunits, CD3γ, CD3δ and CD3ε, in a ratio of 1:1:2. Each of the three subunits can be measured to determine the expression, presence or absence of CD3 as a whole.

This approach serves, for example, for the detection of the presence or absence of T Lymphocytes and B Lymphocytes.

In one embodiment, the expression, presence or absence of
at least one marker selected from the group consisting of CD3, CD4 and CD8, and
at least one marker selected from the group consisting of CD20, CD19 and Pax5
   is determined. These marker pairs are particularly indicative for the presence of TLS. In one particular embodiment, the expression, presence or absence of CD3 and CD20 is determined.

The inventors have surprisingly found out that a tumor which is surrounded, infiltrated or accompanied by a tertiary lymphoid structure (TLS) is more likely to respond on immunotherapy. In particular, the presence of one or more tertiary lymphoid structures is associated with a high likelihood of improved survival of the patient, and/or better clinical outcome, in response to immunotherapy, in particular immune checkpoint inhibition therapy, based on the therapeutic agents and regimens discussed herein elsewhere.

Methods to determine the presence of TLS are disclosed in Sautes Friedman et al (2019), the content of which is incorporated herein by reference for enablement purposes.

According to one embodiment of the invention, the method further comprises the step of
determining, in the same sample from said patient, the density of the TLS.

The term “density of TLS” describes the number of TLS structures per area in a given sample. This can be done in a histological slide, after staining in the sample at least one of the above markers or marker pairs (e.g., CD3 and CD20), or histochemical staining, to count the thus stained structures. For such propose, the tumor issue in the sample can be counterstained with a tumor marker, as disclosed elsewhere herein.

According to one embodiment of the invention, the method further comprises the step of
determining, in the same sample from said patient, the maturity status of the TLS and/or the density of mature TLS.

Tertiary lymphoid structures are subject to maturation. However, the dynamics of TLS maturation have so far remained unclear. Generally, three sequential TLS maturation stages can be described and are characterized by increasing prevalence of follicular dendritic cells and mature B-cells:
[1] Early TLS (E-TLS), composed of dense lymphocytic aggregates without Follicular dendritic cells (FDCs),
[2] Primary follicle-like TLS (PFL-TLS), comprising FDCs but no GC reaction, and
[3] Secondary follicle-like TLS (SFL-TLS), demonstrating an active GC reaction.

According to one embodiment of the invention, the determination of the maturity status of the TLS comprises detecting, on a protein- or mRNA basis,
the expression, presence or absence of at least one marker selected from the group consisting of CD35 and CD23, or
the presence or absence of DC-Lamp⁺ mature dendritic cells (mDCs) cells.

In one embodiment, the expression or presence of CD23 is used as an indicative marker for the presence of mature TLS.

According to one embodiment of the invention, the method further comprises the step of
determining, in the same or a second sample from said patient, the expression status of PD-L1 (programmed cell death ligand 1).

According to one embodiment of the invention, the determination of the expression status of PD-L1 comprises detecting, on a protein basis or mRNA basis, the expression, presence or absence of PD-L1 in the sample.

According to one embodiment of the invention, the determination of the expression status of PD-L1 comprises determination of at least one of
Tumor Proportion Score (TPS)
Immune Cell Score (IC), and/or
Combined Positive Score.

Tumor Proportion Score (TPS, also called TC), as used herein, relates to the percentage of PD-L1 positive tumor cells out of all vital tumor cells, as for example determined with a method comprising an immunoassay (Kulangara et al, 2019).

Immune Cell Score (IC), as used herein, relates to the percentage of the area of PD-L1-positive immune cells from the area of vital tumor cells (i.e., “infiltrated immune cells”), as for example determined with a method comprising an immunoassay.

Combined Positive Score (CPS) as used herein, is essentially a combination of TPS and IC), and relates to the percentage of PD-L1 positive cells including lymphocytes and macrophages from all vital tumor cells.

Preferably an Immunohistochemistry (IHC) assay is used for these purposes. Different IHC assays are currently approved to determine any of the above scores.

PD-L1 IHC 22C3 pharmDx (DAKO 22C3) is a IHC assay using Monoclonal Mouse Anti-PD-L1, Clone 22C3 intended for use in the detection of PD-L1 protein in formalin fixed, paraffin-embedded (FFPE) non-small cell lung cancer (NSCLC), gastric or gastroesophageal junction (GEJ) adenocarcinoma and cervical cancer tissues using EnVision FLEX visualization system on Autostainer Link 48 VENTANA PD-L1 (SP142) assay is an IHC assay using rabbit monoclonal anti-PD-L1 clone SP142 intended for use in the assessment of the programmed death-ligand 1 (PD-L1) protein in tumor cells and tumor infiltrating immune cells in the formalin-fixed, paraffin-embedded (FFPE) tissues. Determination of PD-L1 status is indication-specific and evaluation is based on either the proportion of tumor area occupied by PD-L1 expressing tumor-infiltrating immune cells (% IC) of any intensity or the percentage of PD-L1 expressing tumor cells (% TPS/TC) of any intensity.

VENTANA PD-L1 (SP263) assay is an IHC assay using rabbit monoclonal anti-PD-L1 clone SP263 intended for use in the assessment of the PD-L1 protein in formalin-fixed, paraffin-embedded (FFPE) urothelial carcinoma tissue stained with OptiView DAB IHC Detection Kit on a VENTANA BenchMark ULTRA instrument. PD-L1 status is determined by the percentage of tumor cells with any membrane staining above background (TC or TPS) or by the percentage of tumor-associated immune cells with staining (IC) at any intensity above background.

According to one embodiment of the invention, the determination of the expression status of PD-L1 comprises in situ Hybridization (ISH).

in situ hybridization (ISH) is a type of hybridization that uses a labeled complementary DNA, RNA or modified nucleic acids strand (i.e., probe) to localize a specific DNA or RNA sequence in a portion or section of tissue (in situ) or if the tissue is small enough (e.g., plant seeds, Drosophila embryos), in the entire tissue (whole mount ISH), in cells, and in circulating tumor cells (CTCs). This is distinct from immunohistochemistry, which usually localizes proteins in tissue sections.

RNAScope is a commercially available, fully automated, ISH assay for the detection of RNA in a variety of sample types including formalin fixed paraffin embedded tissue. The RNAScope assay visualises RNA by developing a chromogen to produce small punctate dots which are quantitative and therefore less analytically subjective. RNAScope has been used to detect PD-L1 RNA and has been compared to other assays such as IHC in various indications including small cell lung cancer (SCLC) (Yu et al 2017). Positivity using RNAScope was defined in this study as the presence of 4-10 punctate dots per tumour cell.

RNAScope positivity was compared to IHC positivity, defined as a tumour proportion score (percentage of cells with partial or complete cell membrane staining at any intensity) of greater than 1% using Dako 28-8 and Ventana SP142 antibodies. PD-L1 prevalence using RNAScope and IHC assays were similar (15.5% vs 16.5%). Additionally, RNA ISH has been used to retrospectively investigate clinical response and the extent of RNA expression in comparison to IHC (Yuan et al. 2016). RNAScope and IHC identified similar numbers of PD-L1 positive patients (33.9% vs 35.1% respectively). A positive relationship was observed between PD-L1 mRNA and PD-L1 protein expression, 67.3% of PD-L1 IHC positive patient samples were PD-L1 RNAScope positive and 88.2% of PD-L1 IHC negative patient samples were also PD-L1 RNAScope negative. These data suggest RNA detection using RNAScope may offer an alternative assay to identify patients suitable for treatment with immune therapy, however further optimisation would be required to define a suitable threshold or cutoff for determining PD-L1 high expression.

Scoring algorithms for PD-L1 status using RNAScope have been previously proposed. For example, greater than 10 punctate dots per cell (Shi et al 2017) or greater than 10 dots per cell in greater than 10% of cells have been suggested to be indicative of a cut off between PD-L1 high and PD-L1 low status.

The following table gives an overview over thresholds to determine whether or not a sample is characterized by absent, low, medium or high expression of PD-L1 (programmed cell death ligand 1).

| Expression | | | | ISH (punctate dots |
|---|---|---|---|---|
| status | TPS | IC | CPS | per tumour cell) |
| absent | ≤1 | | ≤1 | 0 |
| low | <10 | | <10 | 1-3 |
| middle | ≥10 | | ≥10 | 4-10 |
| high | ≥50 | >1 | ≥50 | |

According to one embodiment of the invention, the determination of the expression status of PD-L1 comprises in situ PCR

In Situ Polymerase Chain Reaction (in situ PCR) is a powerful method that detects even rare or single-copy number nucleic acid sequences in fresh, frozen or paraffin-embedded cells or tissue sections for the localization of those sequences within the cells. The principle of this method involves tissue fixing (to preserve the cell morphology) and subsequent treatment with proteolytic digestion (to provide access for the PCR reagents to the target DNA). The target sequences are amplified by those reagents and then detected by standard immunocytochemical protocols. in situ PCR combines the sensitivity of PCR or RT-PCR amplification along with the ability to perform morphological analysis on the same sample. Assessment of PD-L1 mRNA with such method has for example been disclosed by Duncan et al (2019), the content of which is herewith incorporated by reference.

According to one embodiment of the invention, in a sample suspected to comprise neoplastic or cancerous tissue, at least one of
a) Presence of TLS in the sample,
b) TLS density of ≥0.005 TLS/mm²,
c) Presence of mature TLS,
d) Density of mature TLS of ≥0.005 TLS/mm², and/or
e) absent or low expression of PD-L1 (programmed cell death ligand 1).
   is considered indicative for
a high likelihood of improved survival of the patient, and/or
better clinical outcome
   in response to an immunotherapy.

In one embodiment, determining the density of the TLS is preferred.

In one further embodiment, determining the density of mature TLS is preferred.

In preferred embodiments, the threshold TLS density which is considered indicative for a high likelihood of improved survival of the patient, and/or better clinical outcome, in response to an immunotherapy is ≥0.005/mm², ≥0.01/mm², ≥0.02/mm², ≥0.03/mm², ≥0.04/mm², ≥0.05/mm², ≥0.06/mm², 0.063/mm², ≥0.07/mm², ≥0.08/mm², ≥0.09/mm², ≥0.1/mm², ≥0.11/mm², ≥0.12/mm², ≥0.13/mm², ≥0.14/mm², ≥0.15/mm², ≥0.16/mm², ≥0.17/mm², ≥0.18/mm², ≥0.19/mm², ≥0.2/mm², ≥0.25/mm², ≥0.3/mm², ≥0.35/mm², ≥0.4/mm², ≥0.45/mm², ≥0.5/mm², ≥0.55/mm², ≥0.6/mm², ≥0.65/mm², ≥0.7/mm², ≥0.75/mm², ≥0.8/mm², ≥0.85/mm², ≥0.9/mm², ≥0.95/mm², ≥1/mm².

In a preferred embodiment, the threshold TLS density which is considered indicative for a high likelihood of improved survival of the patient, and/or better clinical outcome, in response to an immunotherapy, is ≥0.023/mm².

In preferred embodiments, the threshold density of mature TLS which is considered indicative for a high likelihood of improved survival of the patient, and/or better clinical outcome, in response to an immunotherapy is ≥0.005/mm², ≥0.01/mm², ≥0.02/mm², ≥0.03/mm², ≥0.04/mm², ≥0.05/mm², ≥0.06/mm², 0.063/mm², ≥0.07/mm², ≥0.08/mm², ≥0.09/mm², ≥0.1/mm², ≥0.11/mm², ≥0.12/mm², ≥0.13/mm², ≥0.14/mm², ≥0.15/mm², ≥0.16/mm², ≥0.17/mm², ≥0.18/mm², ≥0.19/mm², ≥0.2/mm², ≥0.25/mm², ≥0.3/mm², ≥0.35/mm², ≥0.4/mm², ≥0.45/mm², ≥0.5/mm², ≥0.55/mm², ≥0.6/mm², ≥0.65/mm², ≥0.7/mm², ≥0.75/mm², ≥0.8/mm², ≥0.85/mm², ≥0.9/mm², ≥0.95/mm², ≥1/mm².

In preferred embodiment, the threshold density of mature TLS which is considered indicative for a high likelihood of improved survival of the patient, and/or better clinical outcome, in response to an immunotherapy, is ≥0.03/mm².

In one embodiment, the threshold density of mature TLS should be higher than the general threshold TLS density—which is a counterintuitive finding.

The term “improved survival”, as used herein, refers to a prolonged period of time during which the subject or patient is alive after treatment with a method described herein. Improved survival denotes the greatest probability of staying free of disease progression for an individual suffering from cancer after a particular treatment. It is also used to describe the high percentage of individuals in a group whose disease is likely to remain stable (without showing signs of progression) after a specific period of time, compared to a control group. It is also used to describe the high percentage of individuals in a group whose disease is likely to cure (without showing signs of disease) after a specific period of time, compared to a control group. This parameter can be measured by any of the usual clinical endpoints indicated as “progression-free survival”, “overall survival” and “disease-free survival” used as an indication of the effectiveness of a particular treatment.

It is important to understand that the different steps a)-d) can be performed
at the same time and/or in the same location and/or under the same responsibility, and/or
on the same sample, and/or on different samples and/or on different subsamples of the same sample, and/or
at different points of time, and/or in different locations and/or under different responsibilities.

In case at least two of steps a)-d) are performed under different responsibilities, performing one of the steps alone would still fall under the scope of the patent as contributory infringement.

Further, it is important to understand that in one embodiment, steps a)-c) can be performed in a first sample, and step d) can be performed in a second sample.

In several embodiments, the cancer the patient suffers from or is at risk of developing is at least one of thyroid carcinoma, gastrointestinal stromal tumor, cholangiocarcinoma, pancreatic adenocarcinoma, anal carcinoma, cervical cancer, ovarian cancer, vulvar carcinoma, endometrial carcinoma, cervical cancer, ovarian cancer, vulvar carcinoma, endometrial carcinoma, gastric carcinoma, Non-small cell lung cancer, soft-tissue sarcomas, Bladder cancer, Colorectal cancer, Head and neck, Renal cancer and Breast cancer.

According to one embodiment of the invention, the cancer the patient suffers from or is at risk of developing is a lung cancer or a cancer of a digestive organ.

In one embodiment, said lung cancer is NSCLC. In another embodiment, said lung cancer is SCLC.

In different embodiments, the cancer of a digestive organ is at least one selected from the group consisting of colon cancer, metastatic colon cancer, colorectal cancer or metastatic colorectal cancer, esophageal cancer, metastatic esophageal cancer, gastric cancer and metastatic gastric cancer.

According to one embodiment of the invention, the immunotherapy is a therapy in which an immune checkpoint inhibitor is applied.

According to one embodiment of the invention, the immune checkpoint inhibitor is a binder, inhibitor or antagonist of at least one of CTLA-4, PD-1, PD-L1, LAG 3, TIM3, OX40, 4-1BB and/or TIGIT.

According to one embodiment of the invention, the binder, inhibitor or antagonist is an antibody, or a target binding fragment or derivative thereof.

According to one embodiment of the invention, the antibody is at least one selected from the group consisting of is at least one selected from the group consisting of Ipilimumab (anti-CTLA-4), Nivolumab (anti-PD-1), Pembrolizumab (anti-PD-1), Cemiplimab (anti-PD-1), Spartalizumab (anti-PD-1), Atezolizumab (anti-PD-L1), Avelumab (anti-PD-L1), Durvalumab (anti-PD-L1), Etigilimab (anti-TIGIT), BGB-A1217 (anti-TIGIT) BMS-986207 (anti-TIGIT), AB154 (anti-TIGIT) ASP8374 (anti-TIGIT), MK 7684 (anti-TIGIT), and/or Tiragolumab (anti-TIGIT).

In one embodiment, the immune checkpoint inhibitor is a binder, inhibitor or antagonist of PD-L1.

In one embodiment the immune checkpoint inhibitor is Avelumab (PD-L1). Avelumab is a fully human monoclonal antibody which targets the protein programmed death-ligand 1 (PD-L1). It has received orphan drug designation by the European Medicines Agency (EMA) for the treatment of gastric cancer. The US Food and Drug Administration (FDA) approved it in March 2017 for Merkel-cell carcinoma. The EMA approved it in September 2017 for the same indication. The inventors of the present invention have shown a benefit of Avelumab for patients suffering form or being diagnosed for solid tumors and stratified as disclosed herein.

In other embodiments, the binder, inhibitor or antagonist of PD-L1 is Atezolizumab or Durvalumab.

In one embodiment, the immune checkpoint inhibitor is a binder, inhibitor or antagonist of PD1. In one embodiment the immune checkpoint inhibitor is Nivolumab (PD1). Nivolumab is a fully human monoclonal antibody which targets PD-1. Nivolumab received FDA approval for the treatment of melanoma in December 2014. In April 2015, the Committee for Medicinal Products for Human Use of the European Medicines Agency recommended approval of Nivolumab for metastatic melanoma as a monotherapy. In March 2015, the U.S. FDA approved it for the treatment of squamous cell lung cancer. On 19 Jun. 2015, the European Medicines Agency (EMA) granted a marketing authorization valid throughout the European Union.

In other embodiments, the binder, inhibitor or antagonist of PD-1 is Pembrolizumab, Cemiplimab or Spartalizumab.

According to one embodiment of the invention, the method further comprises the step of
determining, in the sample, at least one of presence, volume, area, size or contour of a tumor tissue or lession, by detecting the presence or absence of a tumor marker or staining the sample histochemically.

Both approaches serve to differentiate tumor form stroma and/or TLS can be both in a qualitative and quantitative fashion. This is also useful to define and quantify the size of tumor lesions.

In one embodiment, one such tumor marker is a keratin, preferably a cytokeratin. Cytokeratins are keratin proteins found in the intracytoplasmic cytoskeleton of epithelial tissue. They are an important component of intermediate filaments, which help cells resist mechanical stress. Expression of these cytokeratins within epithelial cells is largely specific to particular organs or tissues. This they can be used as markers to identify tumor tissue. In another embodiment, one such tumor marker is PSA (prostate specific antigen) or CA 19-9.

This can be done, in one embodiment, by methods comprising an immunoassay, e.g., with the use of an anti-keratin antibody, preferably an anti-cytokeratin antibody.

In one embodiment the histochemical staining is a Haematoxyin and Eosin (H&E) staining.

In one embodiment, the presence, volume, area, size or contour of the tumor tissue or tumor lesion is determined, after application of a tumor marker or histochemical staining, with an image analysis system.

This facilitates the determination of TLS density, by merely dividing the count of TLS by the area size, area or volume of the tumor tissue or tumor lesions.

According to one aspect of the invention, an immune checkpoint inhibitor for use in the (manufacture of a medicament for the) treatment of a patient suffering from, or being diagnosed for, cancer is provided, which cancer is characterized by at least one of
a) Presence of TLS in the sample,
b) TLS density of ≥0.005 TLS/mm²,
c) Presence of mature TLS,
d) Density of mature TLS of ≥0.005 TLS/mm², and/or
e) absent or low expression of PD-L1 (programmed cell death ligand 1).

In one embodiment, determining the density of the TLS is preferred.

In one further embodiment, determining the density of mature TLS is preferred.

According to one aspect of the invention, a method of treating a patient that suffers from or is at risk of developing cancer with an immune checkpoint inhibitor is provided, which cancer is characterized by at least one of
a) Presence of TLS in the sample,
b) TLS density of ≥0.005 TLS/mm²,
c) Presence of mature TLS,
d) Density of mature TLS of ≥0.005 TLS/mm², and/or
e) absent or low expression of PD-L1 (programmed cell death ligand 1).
   wherein said method comprises administration of the immune checkpoint inhibitor in at least one therapeutically effective dosis,

In one embodiment, determining the density of the TLS is preferred.

In one further embodiment, determining the density of mature TLS is preferred.

In preferred embodiments, the threshold TLS density is ≥0.005/mm², ≥0.01/mm², ≥0.02/mm², ≥0.03/mm², ≥0.04/mm², ≥0.05/mm², ≥0.06/mm², 0.063/mm², ≥0.07/mm², ≥0.08/mm², ≥0.09/mm², ≥0.1/mm², ≥0.11/mm², ≥0.12/mm², ≥0.13/mm², ≥0.14/mm², ≥0.15/mm², ≥0.16/mm², ≥0.17/mm², ≥0.18/mm², ≥0.19/mm², ≥0.2/mm², ≥0.25/mm², ≥0.3/mm², ≥0.35/mm², ≥0.4/mm², ≥0.45/mm², ≥0.5/mm², ≥0.55/mm², ≥0.6/mm², ≥0.65/mm², ≥0.7/mm², ≥0.75/mm², ≥0.8/mm², ≥0.85/mm², ≥0.9/mm², ≥0.95/mm², ≥1/mm².

In a preferred embodiment, the threshold TLS density is ≥0.023/mm².

In preferred embodiments, the threshold density of mature TLS is ≥0.005/mm², ≥0.01/mm², ≥0.02/mm², ≥0.03/mm², ≥0.04/mm², ≥0.05/mm², ≥0.06/mm², 0.063/mm², ≥0.07/mm², ≥0.08/mm², ≥0.09/mm², ≥0.1/mm², ≥0.11/mm², ≥0.12/mm², ≥0.13/mm², ≥0.14/mm², ≥0.15/mm², ≥0.16/mm², ≥0.17/mm², ≥0.18/mm², ≥0.19/mm², ≥0.2/mm², ≥0.25/mm², ≥0.3/mm², ≥0.35/mm², ≥0.4/mm², ≥0.45/mm², ≥0.5/mm², ≥0.55/mm², ≥0.6/mm², ≥0.65/mm², ≥0.7/mm², ≥0.75/mm², ≥0.8/mm², ≥0.85/mm², ≥0.9/mm², ≥0.95/mm², ≥1/mm².

In preferred embodiment, the threshold density of mature TLS is ≥0.03/mm².

In one embodiment, the threshold density of mature TLS should be higher than the general threshold TLS density—which is a counterintuitive finding.

According to one embodiment of the invention, the immune checkpoint inhibitor is a binder, inhibitor or antagonist of at least one of CTLA-4, PD-1, PD-L1, LAG 3, TIM3, OX40, 4-1BB and/or TIGIT.

According to one embodiment of the invention, the binder, inhibitor or antagonist is an antibody, or a target binding fragment or derivative thereof.

According to one embodiment of the invention, the antibody is at least one selected from the group consisting of is at least one selected from the group consisting of Ipilimumab (anti-CTLA-4), Nivolumab (anti-PD-1), Pembrolizumab (anti-PD-1), Cemiplimab (anti-PD-1), Spartalizumab (anti-PD-1), Atezolizumab (anti-PD-L1), Avelumab (anti-PD-L1), Durvalumab (anti-PD-L1), Etigilimab (anti-TIGIT), BGB-A1217 (anti-TIGIT) BMS-986207 (anti-TIGIT), AB154 (anti-TIGIT) ASP8374 (anti-TIGIT), MK 7684 (anti-TIGIT), and/or Tiragolumab (anti-TIGIT).

In one embodiment, the immune checkpoint inhibitor is a bispecific binder, e.g., a bispecific antibody.

In one embodiment, the bispecific binder binds CTLA-4 and PD-1 or PD-L1.

In further embodiments, such bispecific binder is at least one selected from the group consisting of KN046 (Alphamab), AK104 (Akeso), XmAb20717 (Xencor), MEDI5752 (Astrazeneca), MDG019 (Macrogenics), CN104974253A (Shanghai CITIC Guojian Pharmaceutical).

In one embodiment, the bispecific binder binds 4-1BB (CD137) and PD-1 or PD-L1.

In further embodiments, such bispecific binder is at least one selected from the group consisting of INBRX-105 (Inhibrx/Elpiscience Biopharmaceuticals), MCLA-145 (Merus), FS222 (F-star).

According to one aspect of the invention, a combination of an immune checkpoint inhibitor with another immune checkpoint inhibitor or a chemotherapeutic drug,

optionally for use in the manufacture of a medicament or in the manufacture of separate coadministrable medicaments
is provided for the treatment of a patient suffering from, or being diagnosed for, cancer, which cancer is characterized by at least one of
a) Presence of TLS in the sample,
b) TLS density of ≥0.005 TLS/mm²,
c) Presence of mature TLS,
d) Density of mature TLS of ≥0.005 TLS/mm², and/or
e) absent or low expression of PD-L1 (programmed cell death ligand 1).
   wherein the combination of the two drugs is administered to a patient concomitantly or consecutively.

In one embodiment, determining the density of the TLS is preferred.

In one further embodiment, determining the density of mature TLS is preferred.

As used herein the term “concomitantly” means that the two drugs are administered to the patient at the same time, yet in the same or in different dosage units.

As used herein the term “consecutively” means that the two drugs are administered to the patient at different times, yet to obtain a synergistic or complimentary effect.

According to one aspect of the invention, a method of treating a patient that suffers from or is at risk of developing cancer is provided with a combination of an immune checkpoint inhibitor with another immune checkpoint inhibitor or a chemotherapeutic drug, which cancer is characterized by at least one of
a) Presence of TLS in the sample,
b) TLS density of ≥0.005 TLS/mm²,
c) Presence of mature TLS,
d) Density of mature TLS of ≥0.005 TLS/mm², and/or
e) absent or low expression of PD-L1 (programmed cell death ligand 1).
   wherein said method comprises administration of the immune checkpoint inhibitor (s) and optionally the chemotherapeutic drug in at least one therapeutically effective dosis.

In one embodiment, determining the density of the TLS is preferred.

In one further embodiment, determining the density of mature TLS is preferred.

In one embodiment, the combination comprises a binder, inhibitor or antagonist of CTLA-4 and a binder, inhibitor or antagonist of PD-1 or PD-L1.

In one embodiment, the combination comprises a binder, inhibitor or antagonist of 4-1BB (CD137) and a binder, inhibitor or antagonist of PD-1 or PD-L1.

With regard to these aspects, to avoid lengthy repetitions, the same considerations apply as disclosed elsewhere herein regarding preferred immune checkpoint inhibitors.

According to one embodiment of the invention, the said cancer is at least one selected from the group consisting of a lung cancer or a cancer of a digestive organ. With regard to this aspect, to avoid lengthy repetitions, the same considerations apply as disclosed elsewhere herein regarding preferred types of tumor.

According to another aspect of the invention, a kit for carrying out a method according to the above description is provided, said kit comprising means for at least one of
a) determining whether or not sample comprises a tertiary lymphoid structure (TLS),
b) determining in a sample the maturity status of the TLS.
c) determining in the same the density of the TLS
d) determining is the sample the density of mature TLS, or
e) determining, in a sample the expression status of PD-L1 (programmed cell death ligand 1).

In one embodiment, determining the density of the TLS is preferred.

In one further embodiment, determining the density of mature TLS is preferred.

According to one embodiment of the invention, the kit comprises at least one oligonucleotide comprising a nucleotide sequence which is capable of hybridizing to a nucleic acid encoding for at least one of PDL1, CD20, CD3, CD23, which oligonucleotide is selected from the group consisting of
an amplification primer
a labelled probe, and/or
a substrate bound probe
   wherein said hybridization occurs under stringent conditions, namely conditions under which a probe or primer will hybridize to its target subsequence, but to no other sequences.

The skilled person is able, with routine methods, to design suitable probes and/or primers for the above markers with respective software that is freely accessible. Such software requires, as input, for example the Entrez Gene ID or RefSeq identifier (see table 1 for the targets of interest), and delivers suitable primer or probe sequences. One such software tool is provided by NCBI under the URL https://www.ncbi.nlm.nih.gov/tools/primer-blast/

According to one embodiment of the invention, kit is suitable for at least one of
Fluorescent in situ hybridization (FISH),
in situ PCR,
Real time PCR, and/or
Reverse transcription PCR/qPCR.

All these methods are well known to the skilled person. While the former two deliver spatial resolution and can hence be used for density or proximity measurements, the latter two don't.

According to one embodiment of the invention, the kit comprises at least one immunoligand capable of binding to at least one of CD3, CD4, CD8, CD20, CD19, Pax5, CD21, CD35, CD23, CD8, DC-Lamp or PD-L1 in an immunoassay.

Further, said kit may comprise at least one further immunoligand capable of binding to at least one of Pan Keratin, CK 20, CK7, CK8/18, CK19, PSA, CA19-9, IDO1

The skilled person is able, with routine methods, to select an immunoligand, e.g., an antibody, for the above markers either from respective catalogues, provided e.g. by Abcam, Merck Millipore or ThermoFisher, or make such an immunoligand, e.g., an antibody, with routine methods, like immunization and hymbridoma technique.

Table 1 shows further characteristics of the markers discussed herein, to enable the skilled person to, inter alia, make or select an antibody or design suitable primers or probes, as disclosed elsewhere herein.

According to one embodiment of the invention, said immunoassay is at least one selected from the group consisting of
IHC (Immunohistochemistry assay)
ICC (Immunocytochemistry assay), and/or
IF (Immunofluorescence assay)

According to one embodiment of the invention, at least one of said immunoligands is an antibody.

Such antibody is optionally labelled, e.g., with a fluorescent label, a radioopaque label, an enzymatic label (like horseradish peroxidase), biotin, or the like.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

### TLS Definition

TLSs were defined as lymphoid aggregates made up of B-cell follicles of at least 100 cells and located either within the tumor bulk or at the invasive margin (defined as fibrous tissue distant of less than 500 μm from tumor cells), as previously described (Petitprez et al, Nature, 2020). The topography of each TLS was systematically assessed and classified as follow “intra-tumor TLS” when the TLS was admixed to tumor cells and “peripheral TLS” when the TLS was located within the margin. When the TLS status was assessed on lymphoid organs, (namely lymph nodes, spleen, tonsils), TLS were only taken into account when admixed to tumor cells and if distant from the residual parenchyma, to exclude pre-existing lymphoid follicles. TLS were defined as early TLS (E-TLS) if negative for both CD21 and CD23, primary follicle-like TLS (“PFL-TLS”) if TLS stained only for CD21 and secondary follicle-like TLS (“SFL-TLS”) when the germinal center was positive for at least CD23 or both markers. With the pathology assessment scheme, the TLS were classified as “mature” when CD23+ follicular dendritic cells were present in the GC or “immature” in their absence.

### Method for Cut-Point Calculation

The identification of the optimal cutpoint for the density of TLS or mature TLS was done by using the surv_cutpoint function from the R package ‘survminer’ (v 0.4.7). This function uses the maximally selected rank statistics from the ‘maxstat’ R package. Survival rates were estimated using the Kaplan-Meier method.

Method to Derive TLS Count from a Picture

There are two methods possible for TLS counting:
1) manual by an expert anatomopathologist
2) automated using image analysis after slide digitization

With respect to the automated image analysis, before image analysis—and in case no specific tumor marker (e.g. Keratins) is being used—manual tissue annotation can be done in order to partition image into tissue regions, e.g. tumor vs others. Such annotation can be done using several softwares (depending on the software that is used for the image analysis) including Phenochart (Akoya). Regions can be drawn using a computer mouse or an interactive pen tool and, ideally, is performed by an expert anatomopathologist. Once the tumor region is defined, its respective surface can be calculated by mean of image analysis—softwares including Inform (Akoya) or Image J can be used.

Using dedicated software which are based on machine learning—where a user can train the software to recognize a particular region of interest (eg. TLS) based on appropriate stainings (eg. CD3/CD20/CD23 for mature TLS or CD3/CD20 for TLS)—specific structures can be automatically detected and counted (see Figures). Combining the TLS count and the tumor surface, density of TLS can be calculated.

### Example 1

### Material & Methods

Patients' cohort: 332 patients (196 males, 136 females) treated with iCPI at Institut Bergonie (Bordeaux, France) between December 2013 and May 2019 entered the study. Median age was 61 years (range 19-88). 132 (39.8%) patients had non-small cell lung cancer (39.8%), 47 (14.2%) sarcomas, 31 (9.3%) bladder, 27 (8.1%) colorectal, 12 (3.6%) head and neck, 11 (3.3%) renal, and 72 (21.7%) other solid tumors. 198 (59.6%) patients received a PD1 antagonist (Pembrolizumab or Nivolumab), 134 (30.1%) a PD-L1 antagonist (Atezolizumab or Durvalumab) and 32 (10.2%) a combination of Immune checkpoint inhibitors (Durvalumab+ Tremelimumab or Nivolumab+Ipilimumab). Median follow-up was 19.1 months.

### Method

We investigated by automated immunohistochemistry the presence of TLS and PD-L1 status in a series of tumor specimens from cancer patients treated with iCPI at Institut Bergonie (Bordeaux, France) between December 2013 and May 2019. TLS were defined as a CD20+ B-cell follicle juxtaposed to a CD3+ T cell. All cases were reviewed blindly by 2 pathologists for the presence or absence of TLS as well as for PDL1 scores (TPS and CPS). Tumor surfaces were quantified after slide digitalization and manual annotation by a pathologist

### Results

Presence of TLS was revealed in 110 patients (33.1%) and was correlated neither with the TPS nor with the CPS score—TPS≥10 in 71 patients (21.4%). Presence of TLS was highly predictive of objective response in patients with low PD-L1 expression (TPS<10): 30.9% versus 12.8%, p<0.0001 whereas no significant difference was observed in patients with higher expression (TPS≥10) 53.3 versus 47.5%, p=0.146). Presence of TLS was also associated with improved PFS (4.1 versus 2.6 months, p=0.024) and OS (28 versus 12.6 months, p=0.001) only in patients with low PD-L1. On multivariate analysis performed on the whole cohort, presence of TLS was an independent prognostic factor of PFS and OS together with PD-L1 expression status and Performance status and represented the most significant predictor of OS. Similar results were obtained by performing an analysis among patients with NSCLC only. Altogether, our results indicate that TLS may represent a new biomarker easily assessable in the routine setting that can be used in combination with PD-L1 expression assays to better tailor ICI in cancer patients.

Presence of TLS was highly predictive of objective response in patients with low PD-L1 expression (TPS<10): 30.9% versus 12.8%, p<0.0001 whereas no significant difference was observed in patients with higher expression (TPS≥10) 53.3 versus 47.5%, p=0.146).

### Immunohistochemistry Analysis

Immunohistochemistry (IHC) analysis was performed on the automated Ventana Discovery XT staining platform (Ventana Medical Systems). Slides were deparaffinised in xylene and hydrated in serial alcohol solutions. Antigen retrieval was performed by heat-induced epitope retrieval method using standard CC1 (tris based buffer) pH8 (Ventana Medical Systems). The slides were incubated with the following primary antibodies: anti-CD3 (clone 2GV6, Roche, ready-to-use) and anti-CD20 (clone L26, Roche, ready-to-use) or anti-CD8 (clone C8/144B, Dako, dilution 1/25) and anti-PDL1 (clone QR1, Dako, dilution 1/100). Bound primary antibodies were detected using either OmniMap anti-Ms or Rb HRP together with Chromo MAP DAB detection kit or Discovery Purple kit (Ventana Medical Systems). The slides were counterstained with hematoxylin and cover-slipped. All cases were reviewed blindly by 2 pathologists for the presence or absence of TLS as well as for PDL1 scores (TPS and CPS). Stained slides were imaged on the multispectral slide analysis system and analyzed in Inform image analysis software (Perkin Elmer, version 2.4.1) in order to quantify tumor surface and then evaluate TLS density but also abundance of CD3 and CD20 positive cells within TLS. Data were then computed in R software using Phenoptr and PhenoptrReports packages.

### Immunohistofluorescence Analysis

Immunohistofluorescence (IHF) analysis was performed on the automated Ventana Discovery XT staining platform (Ventana Medical Systems). Slides were deparaffinised in xylene and hydrated in serial alcohol solutions. Antigen retrieval was performed by heat-induced epitope retrieval method using standard CC1 (tris based buffer) pH8 (Ventana Medical Systems). The slides were incubated with the following primary antibodies: anti-CD8 (clone C8/144B, Dako, dilution 1/25), anti-CD4 (clone 4B12, Dako, dilution 1/100 eme), anti-CD20 (clone L26, Roche, ready-to-use), anti-CD21 (clone 2G9, Roche, ready-to-use), anti-CD23 (clone SP23, Roche, ready-to-use). Bound primary antibodies were detected using either OmniMap anti-Ms or Rb-HRP together with Opal detection kit (Akoya Bioscience). The slides were counterstained with Spectral DAPI (Perkin Elmer) and cover-slipped. Stained slides were imaged on the multispectral slide analysis system (Vectra Polaris, Perkin Elmer) and images were then reviewed blindly by 2 pathologists for the assessment of TLS status—mature vs immature. In parallel, image were analysed using Inform image analysis software (Perkin Elmer, version 2.4.1) in order to segment tissue into TLS tissue and others tissues and to phenotype TLS through the quantification of each cell population. Immunophenotype of TLS were then computed in R software using Phenoptr and PhenoptrReports packages.

### REFERENCES

The disclosures of these documents are herein incorporated by reference in their entireties.
Sautes-Fridman, C., Petitprez, F., Calderaro, J. et al. Nat Rev Cancer 19, 307-325 (2019).
Petiprez et al, Nature volume 577, pages 556-560(2020)
Helmink et al, Nature 2020 January; 577(7791):549-555
Cabrita et al, Nature volume 577, pages 561-565(2020)
Shi X et al, PD-L1 expression in lung adenosquamous carcinomas compared with the more common variants of non-small cell lung cancer. SciRep. 2017; 7:46209
Yuan J et al, Programmed death-ligand-1 expression in advanced gastric cancer detected with RNA in situ hybridization and its clinical significance. Oncotarget. 2016; 7(26):3967 1-9, 96
Yu H et al, PD-L1 Expression by Two Complementary Diagnostic Assays and mRNA in situ Hybridization in Small Cell Lung Cancer. J Thorac Oncol. 2017; 12(1): 110-20.
Duncan D J et al. (2019). PLoS ONE 14(4):e0215393.
Kulangara et al. Arch Pathol Lab Med—Vol 143, March 2019

TABLE 1

| | | | Refseq | | | |
|---|---|---|---|---|---|---|
| | | | genomic | Refseq mRNA | | |
| Target | Alias | Uniprot | (example) | (example) | For the detection of | target can be detected e.g. with |
| CD3 | | | | | Presence of TLS | clone 2GV, Roche |
| CD3γ | | P09693 | NG_007566.1 | XM_005271724.4 | | |
| CD3δ | | P04234 | NG_009891.1 | NM_000732.6 | | |
| CD3ε | | P07766 | NG_007383.1 | NM_000733.4 | | |
| CD4 | | P01730 | NG_027688.1 | NM_000616.5 | | clone 4B12 |
| CD8 | | P01732 | NG_011608.2 | NM_001145873.1 | | clone C8/144B |
| CD20 | | P11836 | NG_023388.1 | NM_021950.3 | | clone L26, Roche |
| CD19 | | P15391 | NG_007275.1 | NM_001178098.2 | | clone HD37, Merck Millipore |
| Pax5 | Paired box 5 | Q02548 | NG_033894.1 | NM_001280547.2 | | clone 1H9, Merck Millipore |
| CD21 | | P20023 | NG_013006.1 | NM_001877.5 | Maturity of TLS | clone 2G9, Roche |
| CD35 | | P17927 | NG_007481.1 | NM_000573.4 | | MA5-13122, Thermo Fisher |
| | | | | | | Scientific |
| CD23 | | P06734 | NG_029554.1 | NM_001207019.2 | | clone SP23, Roche |
| DC-Lamp | LAMP3 | Q9UQV4 | | NM_014398.4 | | DDX0190P-100, Novus bio |
| PD-L1 | Programmed cell | Q9NZQ7 | | NM_001267706.1 | PD-L1 status | clone QR1, Dako |
| | death Ligand 1 | | | | | |
| Pan Keratin | | Antibodies detect different Keratins | | | all types of tumors | Pan Keratin antibody (clone |
| | | | | | | AE1/AE3/PCK26, Roche) |
| CK7 | Keratin 7 | P08729 | NG_012296.1 | NM_005556.4 | Biliary duct/Bladder/Breast/Lung/ | Cytokeratin 19 (SP52) - Roche |
| | | | | | Ovary/Pancreas/Uterus tumor | |
| CK8 | Keratin 8 | P05787 | NG_008402.2 | NM_001256282.2 | Biliary duct/Bladder/Breast/ | anti-cytokeratin CK8/18 |
| CK18 | Keratin 18 | P05783 | NG_008351.1 | NM_000224.3 | Colon/Kidney/Liver/Lung/Ovary/ | (5D3) - Leica |
| | | | | | Pancreas/Pleura/Prostate/ | |
| | | | | | Merkel cell/Stomach/Uterus | |
| | | | | | tumor | |
| CK19 | Keratin 19 | P08727 | NG_012285.1 | NM_002276.5 | Biliary duct/Bladder/Breast/ | Cytokeratin 19 (A53- B/A2.26) - |
| | | | | | Cervix/Colon/Lung/Ovary/ | Roche |
| | | | | | Pancreas/Pleura/Prostate/ | |
| | | | | | Stomach/Uterus tumor | |
| PSA | prostate specific | P07288 | NG_011653.1 | NM_001030047.1 | Prostate tumor | anti-Prostate Specific Antigen |
| | antigen | | | | | antibody (Abcam ab53774) |
| CA19-9 | Carbohydrate | CAS Number 92448-22-1 | | | Pancreas tumor | CA19-9 Antibody (MA5-13275) |
| | antigen 19-9 | | | | | by Thermo Fisher |

1. An in vitro method for the prognosis of disease progression in a patient that suffers from or is at risk of developing cancer, said method comprising the step of:
determining, in a sample from said patient which is suspected to comprise neoplastic or cancerous tissue, whether or not the sample comprises a tertiary lymphoid structure (TLS), and
prognosing disease progression
wherein the prognosis is an assessment regarding the
likelihood of improved survival of the patient, and/or
better clinical outcome of an immunotherapy or treatment with an immunotherapeutic agent.

2. The in vitro method according to claim 1, wherein determination of whether or not the sample comprises a tertiary lymphoid structure comprises at least one of
staining the sample with a histochemical staining, and/or
determining the presence or absence of T Lymphocytes and/or B Lymphocytes in the sample.

3. The in vitro method according to claim 18, wherein the expression, presence or absence of at least one marker selected from the group consisting of CD3, CD4, CD8, CD20, CD19 and Pax5 is detected on a protein- or mRNA basis.

4. The in vitro method according to claim 1, which further comprises the step of
determining, in the same sample from said patient, the density of the TLS.

5. The in vitro method according to claim 1, which further comprises the step of
determining, in the same sample from said patient, the maturity status of the TLS and/or the density of mature TLS.

6. The in vitro method according to claim 5, wherein determination of the maturity status of the TLS comprises detecting, on a protein- or mRNA basis,
the expression, presence or absence of at least one marker selected from the group consisting of CD35 and CD23, or
the presence or absence of DC-Lamp⁺ mature dendritic cells (mDCs) cells.

7. The in vitro method according to claim 1, which further comprises the step of
determining, in the same or a second sample from said patient, the expression status of PD-L1 (programmed cell death ligand 1).

8. The in vitro method according to claim 7, wherein determination of the expression status of PD-L1 comprises detecting, on a protein basis or mRNA basis, the expression, presence or absence of PD-L1 in the sample.

9. The in vitro method according to claim 7, wherein determination of the expression status of PD-L1 comprises determination of at least one of
Tumor Proportion Score (TPS)
Immune Cell Score (IC), and/or
Combined Positive Score.

10. The in vitro method according to claim 7, wherein determination of the expression status of PD-L1 comprises in situ Hybridization (ISH).

11. The in vitro method according to claim 7, wherein determination of the expression status of PD-L1 comprises in situ PCR.

12. The in vitro method according claim 1, wherein in the sample suspected to comprise neoplastic or cancerous tissue, at least one of
a) Presence of TLS in the sample,
b) TLS density of ≥0.005 TLS/mm²,
c) Presence of mature TLS,
d) Density of mature TLS of ≥0.005 TLS/mm², and/or
e) absent or low expression of PD-L1 (programmed cell death ligand 1)
is considered indicative for
a high likelihood of improved survival of the patient, and/or
better clinical outcome in response to an immunotherapy.

13. The in vitro method according to claim 1, wherein the cancer the patient suffers from or is at risk of developing is a lung cancer or a cancer of a digestive organ.

14. The in vitro method according to claim 1, wherein the immunotherapy is a therapy in which an immune checkpoint inhibitor is applied.

15. The in vitro method according to claim 14, wherein the immune checkpoint inhibitor is a binder, inhibitor or antagonist of at least one of CTLA-4, PD-1, PD-L1, LAG 3, TIM3, OX40, 4-1BB and/or TIGIT.

16. The in vitro method according claim 15, wherein the binder, inhibitor or antagonist is an antibody, or a target binding fragment or derivative thereof.

17. The in vitro method according to claim 16, wherein the antibody is at least one selected from the group consisting of Ipilimumab (anti-CTLA-4), Nivolumab (anti-PD-1), Pembrolizumab (anti-PD-1), Cemiplimab (anti-PD-1), Spartalizumab (anti-PD-1), Atezolizumab (anti-PD-L1), Avelumab (anti-PD-L1), Durvalumab (anti-PD-L1), Etigilimab (anti-TIGIT), BGB-A1217 (anti-TIGIT) BMS-986207 (anti-TIGIT), AB154 (anti-TIGIT) ASP8374 (anti-TIGIT), MK 7684 (anti-TIGIT), and Tiragolumab (anti-TIGIT).

18. The in vitro method according to claim 1, which further comprises the step of
determining, in the sample, at least one of presence, volume, area, size or contour of a tumor tissue or lession by detecting the presence or absence of a tumor marker or staining the sample histochemically.

19. A medicament, comprising an immune checkpoint inhibitor for treating a patient suffering from, or being diagnosed for, cancer, which cancer is characterized by at least one of
a) Presence of TLS in the sample,
b) TLS density of ≥0.005 TLS/mm²,
c) Presence of mature TLS,
d) Density of mature TLS of ≥0.005 TLS/mm², and/or
e) absent or low expression of PD-L1 (programmed cell death ligand 1).

20. The medicament according to claim 19, which is administered in at least one therapeutically effective dose.

21. A medicament comprising a combination of an immune checkpoint inhibitor with another immune checkpoint inhibitor or a chemotherapeutic drug, for the treatment of a patient suffering from, or being diagnosed for, cancer, which cancer is characterized by at least one of
a) Presence of TLS in the sample,
b) TLS density of ≥0.005 TLS/mm²,
c) Presence of mature TLS,
d) Density of mature TLS of ≥0.005 TLS/mm², and/or
e) absent or low expression of PD-L1 (programmed cell death ligand 1)
wherein the combination of the two drugs is administered to a patient concomitantly or consecutively.

22. The medicament according to claim 21, which is administered in at least one therapeutically effective dose.

23. The medicament according to claim 21, wherein the combination comprises
a binder, inhibitor or antagonist of CTLA-4 and a binder, inhibitor or antagonist of PD-1 or PD-L1, or
a binder, inhibitor or antagonist of 4-1BB (CD137) and a binder, inhibitor or antagonist of PD-1 or PD-L1.

24. The medicament according to claim 21, wherein the immune checkpoint inhibitor is a binder, inhibitor or antagonist of at least one of CTLA-4, PD-1, PD-L1, LAG 3, TIM3, OX40, 4-1BB and/or TIGIT.

25. The medicament according to claim 23, wherein the binder, inhibitor or antagonist is an antibody, or a target binding fragment or derivative thereof.

26. The medicament according to claim 25, wherein the antibody is at least one selected from the group consisting of Ipilimumab (anti-CTLA-4), Nivolumab (anti-PD-1), Pembrolizumab (anti-PD-1), Cemiplimab (anti-PD-1), Spartalizumab (anti-PD-1), Atezolizumab (anti-PD-L1), Avelumab (anti-PD-L1), Durvalumab (anti-PD-L1), Etigilimab (anti-TIGIT), BGB-A1217 (anti-TIGIT) BMS-986207 (anti-TIGIT), AB154 (anti-TIGIT) ASP8374 (anti-TIGIT), MK 7684 (anti-TIGIT), and Tiragolumab (anti-TIGIT).

27. The in vitro method according to claim 14, wherein the immune checkpoint inhibitor is a bispecific binder.

28. The medicament according to claim 19, characterized in that said cancer is a lung cancer or a cancer of a digestive organ.

29. A kit for carrying out a method according to claim 1, said kit comprising means for at least one of
a) determining whether or not sample comprises a tertiary lymphoid structure (TLS),
b) determining in a sample the maturity status of the TLS
c) determining in the same the density of the TLS
d) determining is the sample the density of mature TLS, or
e) determining, in a sample the expression status of PD-L1 (programmed cell death ligand 1).

30. The Kit according to claim 29, which kit comprises at least one oligonucleotide comprising a nucleotide sequence which is capable of hybridizing to a nucleic acid encoding for at least one of PDL1, CD20, CD3, CD23, which oligonucleotide is selected from the group consisting of
an amplification primer
a labelled probe, and/or
a substrate bound probe
wherein said hybridization occurs under stringent conditions, namely conditions under which a probe or primer will hybridize to its target subsequence, but to no other sequences.

31. The Kit according to claim 29, which kit is suitable for at least one of
Fluorescent in situ hybridization (FISH),
in situ PCR,
Real time PCR, and/or
Reverse transcription PCR/qPCR.

32. The Kit according to claim 29, which kit comprises at least one immunoligand capable of binding to at least one of CD3, CD4, CD8, CD20, CD19, Pax5, CD21, CD35, CD23, CD8, DC-Lamp or PD-L1 in an immunoassay.

33. The Kit according to claim 32, wherein said immunoassay is at least one selected from the group consisting of
IHC (Immunohistochemistry assay)
ICC (Immunocytochemistry assay), and/or
IF (Immunofluorescence assay).

34. The Kit according to claim 32, wherein at least one of said immunoligands is an antibody.

35. The medicament according to claim 21, wherein at least one immune checkpoint inhibitor is a bispecific binder.
